(19) United States
(12) Patent Application Publication
(10) Pub. No.: US 2024/0285516 A1
(43) Pub. Date: Aug. 29, 2024

(54) COMPOSITIONS COMPRISING PROBIOTIC AND POSTBIOTIC STRAINS OF BACTERIA

(71) Applicant: IIAA Limited, Borehamwood (GB)

(72) Inventors: Lorraine PERRETTA, London (GB); Paul NAUDE, London (GB); Tanya MEYER, London (GB)

(21) Appl. No.: 18/652,326

(22) Filed: May 1, 2024

(30) Foreign Application Priority Data
Nov. 3, 2021 (GB) 2115797.9

(51) Int. Cl. A61K 8/99; A61K 8/67; A61K 35/744; A61K 35/745; A61K 35/747; A61Q 5/00; A61Q 19/00

(52) U.S. Cl. CPC A61K 8/99; A61K 8/676; A61K 35/744; A61K 35/745; A61K 35/747; A61Q 5/002; A61Q 19/00

(57) ABSTRACT

The present invention relates to a composition comprising at least one probiotic strain of bacteria and at least one postbiotic strain of bacteria. The present invention also relates to a method of improving the appearance skin and/or hair, and a method of altering the microbiome of the skin of a subject, wherein the method comprises orally administering said composition to a subject in need thereof.

### CROSS-REFERENCE TO RELATED APPLICATIONS

This application is a Continuation of PCT Application No. PCT/GB2022052762, filed on Nov. 2, 2022, which claims the benefit of GB Application No. 2115797.9, filed on Nov. 3, 2021, the entire contents of each are hereby incorporated by reference in their entirety.

### FIELD OF THE INVENTION

The present invention concerns compositions comprising at least one probiotic strain of bacteria and at least one postbiotic strain of bacteria. The invention also concerns methods and uses of such compositions in improving the appearance of the skin and/or hair. The invention also concerns methods and uses of such compositions in altering and improving the microbiome of the skin of a subject.

### BACKGROUND OF THE INVENTION

Humans have long sought to improve the appearance of their skin by cosmetic intervention. Undesirable skin imperfections and characteristics include dry skin, oily skin, wrinkles and fine lines, poor elasticity, peeling or flaky skin, roughness and redness. Of particular interest to consumers is the improvement in or reduction of such imperfections and characteristics, particularly when associated with the skin of the face. One specific area of concern for consumers is the appearance of ageing skin.

Skin ageing is caused by several factors, including hereditary and environmental influences. Such environmental factors include exposure to ultraviolet radiation. Features which are characteristic of the skin ageing process include fine lines and wrinkles, skin dryness, a change in thickness of the skin (i.e. thickening of the skin or thinning of the skin), a reduction in elasticity of the skin, and uneven skin tone.

The ageing process not only impacts on the appearance of skin, but also changes the appearance of the hair, particularly the hair of the scalp. For example, with age, hair may become thinner and more brittle, less dense, less glossy, and may lose some or all of its pigmentation thus becoming grey or white in appearance.

Surgical procedures are available to reduce the visible signs of ageing, particularly of the skin. However, such procedures are invasive and costly and so alternatives are sought.

The human microbiome genome is the combined genetic material of the microorganisms found in and on the human body. Between 2007 and 2013, scientists mapped the microbiome genome to aid the understanding of the role the various microorganisms play and their impact on human health. The human microbiome genome consists of the genes of 10 trillion to 100 trillion symbiotic microbial cells (Ursell et al. Nutr Rev. 2012; 70(Supp1): S38-S44).

Microorganisms play an integral role in maintaining good health. An imbalance of healthy gut microbiota is thought to have an impact on the development of chronic conditions such as obesity, autoimmune disorders, diabetes, chronic fatigue syndrome and non-alcoholic fatty liver disease. More recently, it has been shown that there is an altered gut microbiota in people diagnosed with skin conditions such as atopic dermatitis, psoriasis and rosacea.

Although the mechanisms are not fully understood, a complex relationship is known to exist between gut health and the health of the skin (Salem et al. Frontiers in Microbiology, 2018, 9, Article 459). There is some evidence that metathesis of intestinal microbes and their metabolites to the skin can occur, which alters the skins physiology, pathology, immune response and skin microbiome. The intestinal microbiome also contributes to skin allostasis, which is the restoration of homeostasis after a disturbance or stressor.

The microbial genome can be assessed in different sites of the body. For example, the microbiome may be assessed in the gut (or region thereof) or on the skin (or region thereof). The adult intestine, particularly the lower gut, hosts a diverse range of bacterial, fungal and viral species and is particularly important for overall human health by protecting against diseases, breaking down food to release energy, and also producing essential vitamins. For example, the gut microbiome breaks down indigestible complex polysaccharides and also releases important nutritional components such as vitamin K. Metabolites such as retinoic acid, and polysaccharide A from Bacteroides fragilis, and microbes such as Faecalibacterium prausnitzii and bacteria belonging to the Clostridium cluster IV and XI, promote the accumulation of regulatory T cells which facilitate anti-inflammatory responses.

The gut microbiome is greatly influenced by diet and thus may be altered by the administration of probiotic supplements. Probiotic supplementation is the administration of live beneficial bacteria and/or yeasts to the gut. Probiotic supplements therefore represent a promising option for improving the appearance of skin. However, the field of probiotic supplements, particularly for use in improving the appearance of skin, is in its infancy. There is therefore a need to expand the range of probiotic supplements, and oral supplements in general, to those that can improve the appearance of skin, such as by reducing skin characteristics associated with ageing.

The present invention seeks to mitigate the above-mentioned problems. Alternatively or additionally, the present invention seeks to provide a composition comprising at least one probiotic strain of bacteria and at least one postbiotic strain of bacteria. Alternatively or additionally, the present invention seeks to provide an improved method of improving the appearance of the skin and/or hair by administering a composition comprising a combination of probiotics and postbiotics. Alternatively, or additionally, the present invention seeks to provide an improved method for altering the microbiome of the skin by orally administering a composition comprising a probiotic and postbiotic.

### SUMMARY OF THE INVENTION

According to a first aspect of the invention, there is provided a composition comprising at least one probiotic strain of bacteria, the at least one probiotic strain of bacteria being selected from probiotic strains of Lactobacillus rhamnosus, Pediococcus acidilactici, Bifidobacterium longum, and Lactobacillus reuteri or combination thereof, the composition characterised in that the composition further comprises at least one postbiotic strain of bacteria.

Such a composition may be used to improve the appearance of the skin. In particular, a combination of at least one probiotic strain and at least one postbiotic strain of bacteria may be particularly beneficial in a composition for improving the appearance of the skin. Principally, the composition may reduce or improve the appearance of skin defects, flaws, imperfections or other characteristics associated with ageing. It will be understood that such defects, flaws, imperfections or other characteristics relate to the cosmetic appearance of the skin.

Alternatively, or additionally, the composition may be used to improve the appearance of the hair. Principally, the composition may reduce or improve the appearance of hair defects, flaws, imperfections or other characteristics associated with ageing. It will be understood that such defects, flaws, imperfections or other characteristics relate to the cosmetic appearance of the hair.

Optionally, the at least one postbiotic strain of bacteria is tyndallized. Tyndallization processes may be used to kill bacteria by exposing the bacteria to an elevated temperature so that the bacteria is inactivated but the cell wall remains intact. To ensure that the bacteria cell wall remains intact, the tyndallisation process uses an elevated temperature that does not exceed 100° C.

Preferably, the at least one postbiotic strain of bacteria comprises a postbiotic strain of Lactobacillus acidophilus or Lactobacillus plantarum, or combination thereof. These particular strains of postbiotic bacteria may be particularly advantageous in improving the appearance of the skin and/or hair. It may be that a postbiotic strain of Lactobacillus acidophilus and/or Lactobacillus plantarum may be particularly advantageous in improving the appearance of the skin and/or hair in combination with at least one probiotic strain of Lactobacillus rhamnosus, Pediococcus acidilactici, Bifidobacterium longum, and Lactobacillus reuteri or combination thereof.

Preferably, the at least one probiotic strain of bacteria comprises a probiotic strain of Lactobacillus rhamnosus and/or Lactobacillus reuteri, and the at least one postbiotic strain of bacteria comprises a postbiotic strain of Lactobacillus acidophilus. Optionally, the at least one probiotic strain of bacteria further comprises a probiotic strain of Pediococcus acidilactici or Bifidobacterium longum or mixtures thereof. Optionally, the at least one postbiotic strain of bacteria further comprises a postbiotic strain of Lactobacillus plantarum.

Preferably, the at least one probiotic strain of bacteria comprises a probiotic strain of each of Lactobacillus rhamnosus, Lactobacillus reuteri, Pediococcus acidilactici and Bifidobacterium longum, and the at least one postbiotic strain of bacteria comprises a postbiotic strain of each of Lactobacillus acidophilus and Lactobacillus plantarum. A combination of strains from these species of probiotic and postbiotic bacteria may be particularly advantageous in improving the appearance of the skin and/or hair.

In some embodiments, at least one probiotic strain of bacteria selected from the list of Lactobacillus rhamnosus, Lactobacillus reuteri, Pediococcus acidilactici and Bifidobacterium longum and at least one postbiotic strain of bacteria selected from the list of Lactobacillus acidophilus and Lactobacillus plantarum are beneficial for the appearance of the skin. In some embodiments, at least one probiotic strain of bacteria selected from the list of Lactobacillus rhamnosus, Lactobacillus reuteri, Pediococcus acidilactici and Bifidobacterium longum and at least one postbiotic strain of bacteria selected from the list of Lactobacillus acidophilus and Lactobacillus plantarum are beneficial for the appearance of the hair. It may be that some of the probiotic strains of bacteria and postbiotic strains of bacteria are beneficial to the appearance of the skin, whilst other probiotic strains of bacteria and postbiotic strains of bacteria are beneficial to the appearance of the hair. Preferably, the benefit to the skin and/or hair includes reducing, preventing or slowing the onset of the appearance of ageing skin and/or hair. Such improvements are described herein.

Preferably, the at least one postbiotic strain of bacteria comprises a postbiotic strain of Lactobacillus acidophilus, most preferably wherein the postbiotic strain of Lactobacillus acidophilus is HA-122. Preferably, the at least one postbiotic strain of bacteria comprises a postbiotic strain of Lactobacillus plantarum, most preferably wherein the postbiotic strain of Lactobacillus plantarum is HA-119. Preferably, the at least one probiotic strain of bacteria comprises a probiotic strain of Lactobacillus rhamnosus, most preferably wherein the probiotic strain of Lactobacillus rhamnosus is GG. Preferably, the at least one probiotic strain of bacteria comprises a probiotic strain of Pediococcus acidilactici, most preferably wherein the probiotic strain of Pediococcus acidilactici is HA-524. Preferably, the at least one probiotic strain of bacteria comprises a probiotic strain of Bifidobacterium longum, most preferably wherein the probiotic strain of Bifidobacterium longum is Rosell-175. Preferably, the at least one probiotic strain of bacteria comprises a probiotic strain of Lactobacillus reuteri, most preferably wherein the probiotic strain of Lactobacillus reuteri is HA-188. These particular postbiotic strains of Lactobacillus acidophilus and Lactobacillus plantarum, and particular probiotic strains of Lactobacillus rhamnosus, Pediococcus acidilactici, Bifidobacterium longum and Lactobacillus reuteri may be particularly advantageous for improving the appearance of the skin and/or hair over other postbiotic strains of Lactobacillus acidophilus and Lactobacillus plantarum, and particular probiotic strains of Lactobacillus rhamnosus, Pediococcus acidilactici, Bifidobacterium longum and Lactobacillus reuteri. In some embodiments, one or more of these particular strains of postbiotic bacteria are advantageous in improving the appearance of the skin, particularly in reducing, preventing or slowing the onset of the appearance of ageing skin. Such characteristics of ageing skin are described herein. In some embodiments, one or more of these particular strains of postbiotic bacteria are advantageous in improving the appearance of the hair, particularly in reducing, preventing or slowing the onset of the appearance of ageing hair. Such characteristics of ageing hair are described herein.

Preferably, the composition further comprises at least one vitamin. Preferably the at least one vitamin is selected from the list of vitamin A, vitamin C (ascorbic acid), vitamin D, vitamin E, vitamin K, vitamin B1, vitamin B2, vitamin B3, vitamin B5 (pantothenic acid), vitamin B7 (biotin), vitamin B6, vitamin B12 and vitamin B9 (folate). Vitamin C may be particularly advantageous in the composition of the invention.

Preferably, the at least one vitamin is present in compositions according to embodiments of the invention in an amount equivalent to the recommended daily allowance (RDA) of that vitamin according to age and sex. Alternatively, the at least one vitamin is present in compositions according to embodiments of the invention in an amount equivalent to a fraction or percentage of the recommended daily allowance dose, for example the at least one vitamin may be present in an amount less than the RDA. For example, the at least one vitamin is present in compositions according to embodiments of the invention in an amount of 75%, 50%, or 25% of the RDA of that vitamin according to age and sex. The at least one vitamin may be present in the composition in an amount of between about 0.1 mg and 80 mg per vitamin. For example, between about 5 mg and 25 mg per vitamin. In some embodiments the age and sex of the recipient may not be known and so the vitamin amounts may be calculated as a percentage (for example one of the percentages recited above) of the RDA of a representative adult or a typical, intended or targeted consumer (for example a 20 year old male or a 30 year old female)

Preferably, the composition comprises vitamin C and the amount of vitamin C in the composition is between about 0.1 mg and 80 mg, such as between about 5 mg and 25 mg of vitamin C. Most preferably, the composition comprises about 12 mg of vitamin C.

Preferably, the composition further comprises at least one additional component selected from the list of: a yeast, fungi, enzyme, botanical (for example, green tea), mineral, prebiotic molecule, compound or composition (for example, fructooligosaccharides), and a postbiotic molecule, compound, composition, cell or cell component. The yeast may for example be of the genus Saccharomyces. It will be understood that a botanical is a substance obtained from a plant. It will be understood that a prebiotic molecule, compound or composition is a non-digestible molecule, compound or composition that promotes the growth of beneficial microorganisms in the intestines. It will be understood that a postbiotic molecule, compound or composition is a metabolite of probiotics, or the components that result from probiotic activity in the gut, for example fermentation. A postbiotic may be a cell component or metabolite of a cell. It may be that a postbiotic molecule is a metabolite of a bacterium. It will be understood that a postbiotic cell is an inactivated microbial cell with or without metabolites or cell components that contribute to demonstrated health benefits.

Preferably, the total amount of strains of Bifidobacterium longum in the composition is greater than about 1×108 but less than about 20×109 CFU at the point of manufacture. Most preferably, the total amount of strains of Bifidobacterium longum in the composition is about 5.40×109 CFU at the point of manufacture.

Preferably, the total amount of strains of Lactobacillus rhamnosus in the composition is greater than about 1×108 but less than about 20×109 CFU at the point of manufacture. Most preferably, the total amount of strains of Lactobacillus rhamnosus in the composition is about 5.40×109 CFU at the point of manufacture.

Preferably, the total amount of strains of Pediococcus acidilactici in the composition is greater than about 1×108 but less than about 20×109 CFU at the point of manufacture. Most preferably, the total amount of strains of Pediococcus acidilactici in the composition is about 5.40×109 CFU at the point of manufacture.

Preferably, the total amount of strains of Lactobacillus reuteri in the composition is greater than about 1×108 but less than about 20×109 CFU at the point of manufacture. Most preferably, the total amount of strains of Lactobacillus reuteri in the composition is about 0.165×109 CFU at the point of manufacture.

Preferably, the total amount of strains of Lactobacillus acidophilus in the composition is greater than about 1 mg but less than about 10 mg at the point of manufacture. Most preferably, the total amount of strains of Lactobacillus acidophilus in the composition is about 3.35 mg at the point of manufacture.

Preferably, the total amount of strains of Lactobacillus plantarum in the composition is greater than about 1 mg but less than about 10 mg at the point of manufacture. Most preferably, the total amount of strains of Lactobacillus plantarum in the composition is about 3.3.5 mg at the point of manufacture. The total amount of strains are recited above at the point of manufacture. Following manufacture, for example during storage and distribution, the number of viable cells would be expected to gradually decline. A person skilled in the art would be able to use knowledge of the rate of decline in viability to calculate the approximate number of viable cells present at the point of manufacture. In some embodiments, the rate of decline of viability is less than 5%, 10%, 15% or 20% per months from the point of manufacture.

According to a second aspect of the invention is provided a capsule or powder for oral administration comprising the composition according to any aspect of the invention, especially in reference to the first aspect of the invention. Advantageously, providing the composition according to the present invention in a capsule or powder provides a convenient way of orally administering the composition.

Optionally, the capsule or powder comprises a biologically compatible filler.

According to a third aspect of the invention is provided a liquid for oral administration comprising the composition according to any aspect of the invention, especially in reference to the first aspect of the invention. Advantageously, providing the composition according to the present invention in a liquid provides a convenient way of orally administering the composition For example, the liquid may be in the form of a drink.

According to a fourth aspect of the invention there is provided a method of improving the appearance of the skin and/or hair of a subject, wherein the subject is a human, the method comprising:
(i) orally administering to the subject the composition according to any aspect of the invention, especially in reference to the first aspect of the invention, and,
(ii) repeating step (i) until a cosmetically beneficial improvement in the appearance of the skin and/or hair of the subject has occurred. Advantageously, the improvement in the appearance of the skin and/or hair is an improvement in the cosmetic appearance of the skin and/or hair, particularly an improvement in features of the skin and/or hair associated with ageing.

In some embodiments, the method according to the fourth aspect of the invention improves the appearance of the skin. In other embodiments, the method according to the fourth aspect of the invention improves the appearance of the hair. In some embodiments, the method according to the fourth aspect of the invention improves both the appearance of the skin and the appearance of the hair. Preferably, the improvement in the appearance of the skin and/or hair relates to an improvement in the visible characteristics associated with ageing of the skin and/or hair, as described herein.

A composition relating to all aspects of the invention may preferably be in the form of a tablet, capsule, powder, liquid, drink or food for oral administration. Such forms of the composition will be easy to administer to a subject or by the subject and can be consumed in both a clinical or non-clinical setting. Preferably, the compositions is orally administered in step (i) of the fourth aspect of the invention in the form of a tablet, capsule, powder, liquid, food or drink. Such forms are easily and conveniently administered orally.

Preferably, the composition of the invention is orally administered. The composition may be orally administered without a meal, but most preferably the composition is administered together with a meal. Preferably, the meal to be consumed together with the composition comprises fats, proteins and carbohydrates.

Optionally, the subject is female. Alternatively, the subject is male.

Preferably, the subject is at least 30 years old. The subject may be over 60 years old. Most preferably, the subject is between 30 and 60 years old. It may be that an improvement in the appearance of the skin may be most pronounced in subjects between the age of 30 and 60 years old.

Preferably, administration of the composition may be repeated once a day until a cosmetically beneficial improvement in the appearance of the skin of the subject has occurred.

Preferably, the method further comprises the step of:
(iii) further repeating step (i) in order to maintain the cosmetically beneficial improvement in the appearance of the skin and/or hair of the subject. For example step (i) is repeated once a day for several months, for example three months.

It will be understood that step (i) may be repeated for as long as necessary for there to be observed an improvement in the appearance of the skin and/or hair of a subject. Step (i) may also be repeated as long as necessary to maintain the improvement in the skin and/or hair. Furthermore, step (i) may be repeated after a pause in administration of the composition, for example if the subject forgets to take the composition for several days, or if a significant improvement in the appearance of the skin and/or hair is maintained for several months. Step (i) may be repeated if after pausing the administration of the composition, the subject experiences a decline in the appearance of the skin and/or hair. In such a scenario, repeating step (i) may lead to a return of the skin to the improved condition or appearance.

Preferably, the cosmetically beneficial improvement of the skin is one or more improvement selected from the list of: increased hydration, a reduction in transepidermal water loss, a reduction in skin roughness or an increase in skin smoothness, a reduction in scaling of the skin, a reduction in the appearance of wrinkles and fine lines, such as a reduction in wrinkle depth, length or density, an increase in elasticity, an improvement in erythema, a reduction in melanogenesis such that the skin has a more even colour tone, an increase in skin firmness, a decrease in fatigability, a normalisation of sebum secretion, a decrease in spots, such as the frequency in spot breakouts, or skin imperfections. It will be understood that any characteristic of ageing skin may be improved according to the method of the fourth aspect of the invention. Many of the improvements may result in a softer, smoother feel and appearance of the skin.

It will be understood that skin hydration refers to the water content contained within the skin. For example, skin with poor hydration may feel dry or rough. It will be understood that transepidermal water loss results in a loss of moisture from the skin. Thus, a reducing in transepidermal water loss may result in smoother feeling skin. Skin which has aged may appear rough to the touch or by sight. Opposed to this, healthy, and in particular young skin, may appear smooth to the touch or by sight. Thus, an improvement in the appearance of the skin may result in a decrease in skin roughness and an increase in skin smoothness.

It will be understood that scaling of the skin refers to flaking, peeling or broken areas of skin. Scaly skin may be described as having a pattern akin to a crocodile skin effect. An improvement in the appearance of the skin may result in a decrease in the scaling of the skin such that the amount of flaking, peeling or broken areas is reduced.

Wrinkles and fine lines are a feature commonly associated with aged skin. The improvement in the appearance of the skin may result in a reduction in the appearance of such wrinkles and fine lines, such as a reduction in the depth, length or density per unit area of skin of wrinkles and fine lines.

An improvement may be observed in the elasticity of the skin. This means that the ability of the skin to stretch but then return to its original form is improved.

It will be understood that erythema is a superficial reddening of the skin, usually in patches, as a result of injury or irritation causing dilation of the blood capillaries. The method according to the fourth aspect of the invention may improve the appearance of erythema.

A common feature associated with aged skin is an unevenness in skin tone. It will be understood that a reduction in melanogenesis results in a more even skin tone.

The method according to the fourth aspect of the invention may increase skin firmness.

It will be understood that fatigability refers to the susceptibility of skin to fatigue with age, for example by losing strength. It may be that the method according to the fourth aspect of the invention decreases the fatigability of the skin.

Sebum is an oily secretion of the sebaceous glands. It may be that the method according to the fourth aspect of the invention normalises sebum concentrations if the sebum concentration is either greater or less than normal before treatment.

It may be that the method according to the fourth aspect of the invention decreases the number of spots appearing on the skin, for example the density of spots per area of skin, or the frequency of spot outbreaks may be decreased. It may be that the method according to the fourth aspect of the invention decreases general skin imperfections.

Preferably, the cosmetically beneficial improvement of the hair is one or more improvement selected from the list of: a decrease in hair breakage, an increase in hair thickness, a decrease in hair loss or thinning, a decrease in the loss of hair pigmentation, and an increase in glossy appearance of the hair. The frequency of hair breakage tends to increase with age. It may be that the method according to the fourth aspect of the invention decreases the frequency of hair breakage. Hair may become thinner with age, both in terms of the thickness of individual hairs and also the density of hair produced from skin follicles. It may be that the method according to the fourth aspect of the invention improves the appearance of the hair so that it appears stronger, more voluminous and/or thicker. For example, the individual hairs may become thicker. Alternatively or additionally, it may be that the density of hair increases. A very common change in hair condition associated with ageing is a loss in hair pigmentation. This may result in hair that appears grey or white in colour. It may be that the method according to the fourth aspect of the invention decreases the loss of hair pigmentation, for example so that the hair appears more vibrant in colour.

It may be that the method according to the fourth aspect of the invention can be used to prevent degradation of the skin and/or hair before the ageing process has become visible, or to slow the onset or progression of imperfections associated with ageing. Thus, according to a fifth aspect of the invention is provided a method of preventing the degradation of the skin and/or hair before the ageing process has begun or is obvious visually, or at least slow the onset or progression of imperfections associated with ageing skin. For example, the method according to the fifth aspect of the invention may prevent, or decrease the rate of onset or progression of at least one of the following imperfections associated with ageing skin: decreased hydration, an increase in transepidermal water loss, an increase in skin roughness or an decrease in skin smoothness, an increase in scaling of the skin, an increase in the appearance of wrinkles and fine lines, such as an increase in wrinkle depth, length or density, a decrease in elasticity, a deterioration in erythema, an increase in melanogenesis such that the skin has a less even colour tone, a decrease in skin firmness, an increase in fatigability, a change in sebum secretion from normal, an increase in spots, such as the frequency of spot breakouts, or skin imperfections. The method according to the fifth aspect of the invention may prevent or decrease the rate of onset or progression of at least one of the following imperfections associated with ageing hair: an increase in hair breakage, a decrease in hair thickness, an increase in hair loss or thinning, an increase in the loss of hair pigmentation, and a decrease in glossy appearance of the hair.

The method according to the fifth aspect of the invention may include any method step of the fourth aspect of the invention, except that those method steps are performed before signs of ageing of the skin and/or hair are visible, or at least when the signs of ageing of the skin and/or hair are minimal. For example, the method according to the fifth aspect of the invention may include the steps of:
(i) orally administering to the subject the composition according to any aspect of the invention, especially in reference to the first aspect of the invention, and,
(ii) repeating step (i) to maintain the appearance of the skin and/or hair of the subject.

Advantageously, administering compositions of the invention to the subject maintains the appearance of youthful skin and/or hair. In other words, the method according to the fifth aspect of the invention prevents, delays the onset of or slows the progression of skin and/or hair characteristics associated with ageing. Such skin and hair characteristics associated with ageing are described herein.

In some embodiments, the method according to the fifth aspect of the invention maintains the appearance of the skin. In other embodiments, the method according to the fifth aspect of the invention maintains the appearance of the hair. In some embodiments, the method according to the fifth aspect of the invention maintains both the appearance of the skin and the appearance of the hair. Preferably, the maintenance in the appearance of the skin and/or hair relates to maintenance of the visible characteristics associated with the age of the skin and/or hair, as described herein.

The subject may be a subject according to the fourth aspect of the invention. Preferably, the subject is less than 50 years old, more preferably less than 40 years old.

Preferably, the method according to the fifth aspect of the invention further comprises the step of:
(iii) further repeating step (i) in order to maintain the appearance of the skin and/or hair of the subject, for example step (i) is repeated once a day for several months, for example three months.

It may be that step (iii) is repeated for several years, for example for 2, 4, 6 or 10 years.

Methods of the fourth or fifth aspect of the invention may be methods of altering the skin microbiome. Alteration of the skin biome can lead to an improvement in the appearance of the skin of a subject due to the complex relationship between the microbiome of the skin and skin health and condition.

According to a sixth aspect of the invention there is provided a method of altering the microbiome of the skin of a subject, wherein the subject is a human, the method comprising:
(i) orally administering to the subject the composition according to any other aspect of the invention, especially in reference to the first aspect of the invention, and,
(ii) repeating step (i) until the microbiome of the skin of the subject has altered.

Methods of this aspect of the invention may also be methods of improving the appearance of the skin. Alteration of the skin biome can lead to an improvement in the appearance of the skin of a subject due to the complex relationship between the microbiome of the skin and skin health and condition.

It will be understood that step (i) may be repeated for as long as necessary for the microbiome of the skin of a subject to change. Step (i) may also be repeated as long as necessary to maintain the change in the microbiome of the skin. Furthermore, step (i) may be repeated after a pause in administration of the composition, for example if the subject forgets to take the composition for several days, or if a significant change of the microbiome of the skin is maintained for several months. Step (i) may be repeated if after pausing the administration of the composition, the subject experiences a decline in the microbiome of the skin, for example an undesirable change in the microbiome of the skin. In such a scenario, repeating step (i) may lead to a return of the skin to the improved skin microbiome.

Preferably, altering the microbiome of the skin comprises altering the diversity and/or composition of the microbiome. For example, altering the microbiome of the skin may comprise improving the microbiome of the skin, for example reducing the number of, and/or diversity of, harmful strains of bacteria on the skin. Additionally, or alternatively, improving the microbiome of the skin may increase the number of, and/or diversity of, beneficial strains of bacteria on the skin. It will be understood that reference to the number of strains of bacteria means the population of a particular strain of bacteria, such that reducing the number of harmful strains of bacteria means reducing the population of harmful strains of bacteria and increasing the number of beneficial strains of bacteria means increasing the population of beneficial strains of bacteria. It will be understood that reference to the diversity of strains of bacteria means the number of different types of distinct strains of bacteria, such that reducing the diversity of the harmful strains of bacteria means reducing the number of distinct strains of harmful bacteria and increasing the diversity of the beneficial strains of bacteria means increasing the number of distinct strains of beneficial bacteria. For example, an increase in the number of beneficial strains of bacteria may mean increasing the population of a particular strain of beneficial bacteria by at least 5%, 10%, 20%, 40%, 60%, 80% or 100% compared to before the composition of the invention was administered. A decrease in the number of harmful strains of bacteria may mean decreasing the population of a particular strain of harmful bacteria by at least 5%, 10%, 20%, 40%, 60%, 80% or 100% compared to before the composition of the invention was administered. An increase in the diversity of beneficial strains of bacteria may mean increasing the number of distinct strains of beneficial bacteria by at least 5%, 10%, 20%, 40%, 60%, 80% or 100% compared to before the composition of the invention was administered. A decrease in the diversity of harmful strains of bacteria may mean decreasing the number of distinct strains of harmful bacteria by at least 5%, 10%, 20%, 40%, 60%, 80% or 100% compared to before the composition of the invention was administered.

Preferably, the skin according to any aspect or embodiment of the invention is the skin of the face, scalp, neck, décolletage, back, arms, legs, torso, hands and/or feet of the subject, preferably wherein the skin is the skin of the face of the subject.

Compositions according to any aspect of the invention, especially in relation to the first aspect of the invention, may be used in methods of medical treatment, especially in methods of treating skin diseases. Compositions according to any aspect of the invention, especially in relation to the first aspect of the invention, may be used in methods of treating any condition listed in a further aspect of the invention, especially in relation to the fourth or fifth aspect of the invention. Compositions according to any aspect of the invention, especially in relation to the first aspect of the invention, may be for use as a medicament.

It will of course be appreciated that features described in relation to one aspect of the present invention may be incorporated into other aspects of the present invention. For example, the method of the invention may incorporate any of the features described with reference to the apparatus of the invention and vice versa.

### DESCRIPTION OF THE DRAWINGS

Embodiments of the present invention will now be described by way of example only with reference to the accompanying drawings of which:

FIG. 1 are photographs of the eye area of a participant of Group 2 taken before and after 12 weeks of consuming Formula B. A reduction in wrinkle depth and the number of wrinkles in the eye area was observed by the end of the 12 week trial study;

FIG. 2 are photographs of the eye area of a participant of Group 2 taken before and after 12 weeks of consuming Formula B. A reduction in wrinkle depth and the number of wrinkles in the eye area was observed at the end of the 12 week trial study;

FIG. 3 are images of the cheek area of a participant in Group 2 taken with a Canfield's VISIA skin analysis imaging system before and after 12 weeks of consuming Formula B. A reduction in skin redness (erythema) was observed after the 12 week trial study;

FIG. 4 are images of the cheek area of a participant in Group 2 taken with Canfield's VISIA skin analysis imaging system before and after 12 weeks of consuming Formula B. A reduction in skin redness (erythema) was observed after the 12 week trial study;

FIG. 5 are photographs of the lip area of a participant in Group 2 taken before and after 12 weeks of consuming Formula B. After the 12 week study, the skin of the lip area appeared smooth and had fewer blemishes compared to the image taken at the start of the trial study;

FIG. 6 are photographs of the cheek area of a participant in Group 2 taken before and after 12 weeks of consuming Formula B. A reduction in wrinkle depth and the number of wrinkles in the eye area was observed at the end of the 12 week trial;

FIG. 7 are photographs of the hair and scalp of a participant in Group 2 taken before and after 12 weeks of consuming Formula B. The scalp appeared less dry, with less flaking of the skin at the end of the 12 week trial study; and,

FIG. 8 are photographs of the eyelashes of a participant (SC212SYB6 of Table 5) in Group 2 taken before and after 12 weeks of consuming Formula B. An improvement in eyelash length, volume and grade was observed at the end of the 12 week study trial.

### DETAILED DESCRIPTION

### Compositions

Compositions according to the invention comprise a combination of probiotic strains and postbiotic strains of bacteria. A combination of probiotic and postbiotic strains of bacteria may result in an improvement in the appearance of the skin and/or hair in a subject administered said composition. A combination of probiotic and postbiotic strains of bacteria in a single composition may improve efficacy over a composition comprising only probiotic strains of bacteria, or a composition comprising only postbiotic strains of bacteria.

The International Scientific Association for Probiotics and Prebiotics defines a postbiotic as a “preparation of inanimate microorganisms and/or their components that confers a health benefit on the host ‘Nature reviews gastroenterology & hepatology, volume 18, pages 649-667 (2021))’. The term postbiotic encompasses both metabolites of microorganisms and inanimate microorganisms themselves, such as inanimate bacteria. When used in the context of a “postbiotic strain”, the term postbiotic refers to postbiotic microorganism. In other uses, the term postbiotic refers to postbiotic metabolites, such as molecules, compounds of compositions produced by bacteria. The term parabiotic (alternatively paraprobiotic) may be used to describe a sub-group of postbiotics. That is, the term parabiotic refers to inactivated microbial cells of probiotics (intact or ruptured containing cell components such as peptidoglycans, teichoic acids, surface proteins, etc.) or crude cell extracts (i.e. with complex chemical composition) (Microbioal Cell Factories 19, Article number: 168 (2020), Postbiotics-parabiotics: the new horizons in microbial biotherapy and functional foods, Nataraj et al). It will be understood that when used in the context of a “postbiotic strain” the term postbiotic refers to postbiotic microorganisms and is thus equivalent to and interchangeable with the term parabiotic.

Postbiotic microorganisms, for example postbiotic bacteria may be prepared by killing live bacteria. Any suitable method may be used to kill live microorganisms for example autoclaving or gamma-irradiation. In some embodiments heat-killing may be preferred. It may be preferred that live microoganisms are killed by a process commonly known as tyndallisation. Accordingly, it will be understood that within the context of the present disclosure, a strain of postbiotic bacteria is a bacterial strain that is inanimate, disabled or killed. Preferably, the postbiotic bacterial strain is tyndallised, that is it has been killed by treatment with heat in a tyndallisation process. Various tyndallisation protocols exist, but essentially they involve heating to a temperature below the temperature that would normally be used in an autoclaving process. Autoclaving may involve heating to about 121° C. for 15 minutes and is effective in killing bacterial cells and bacterial spores. It may however produce degradation of the cells to such an extent that beneficial chemical properties thereof are lost. In contrast tyndallisation may involve heating to 100° C. (or just a little below) and holding it there for at least 15 minutes. For example, in a tyndallisation process, bacteria may be heated to a temperature in the range from 60° C. to 100° C., from 80° C. to 100° C., or from 90° C. to 100° C. The bacteria may be heated for at least 15 minutes, for at least 30 minutes or for at least 1 hour. Such a temperature is sufficient to kill microbial cells but may not kill all spores. Tyndallisation repeats this heating and holding process (for example at least 3 times) on consecutive days so that any spores which have survived an earlier heating germinate and are killed in a subsequent heating. Tyndallisation may require heating bacteria in aqueous solution.

On the other hand, probiotics are live microorganisms that contribute to host benefits such as intestinal health. Probiotic strains should remain alive, after passing through the stomach and the upper gastrointestinal tract, until they reach their target site of action, starting from the lower small intestine and further along the digestive system. Thus, it will be understood that within the context of the present disclosure, a strain of probiotic bacteria is a strain in a viable form.

### Probiotic Strains:

Compositions according to the invention comprise at least one probiotic strain of bacteria being selected from probiotic strains of Lactobacillus rhamnosus, Pediococcus acidilactici, Bifidobacterium longum, and Lactobacillus reuteri, or a combination thereof. These microbes are preferably provided in a substantially viable form.

In embodiments of the invention, the at least one probiotic strain of bacteria comprises a probiotic strain of Lactobacillus rhamnosus. Preferably, the strain of Lactobacillus rhamnosus originates from the human microflora. For example, a strain of Lactobacillus rhamnosus may be obtained from the human gut.

In embodiments of the invention, the probiotic strain of Lactobacillus rhamnosus is GG. As an example, Lactobacillus rhamnosus GG may be supplied by Lallemand under the tradename LGG. Lactobacillus rhamnosus GG has been deposited at the Collection Nationale de Cultures de Microorganismes under deposit number I-4798, CNCM. Lactobacillus rhamnosus GG has a cell morphology comprising rods; non-sporulating; non-motile, sometimes curved, isolated, in short chains; Grampositive, facultatively heterofermentative, anaerobe facultative. Lactobacillus rhamnosus GG is an example of a probiotic strain that produces L(+) lactic acid. In laboratory conditions, Lactobacillus rhamnosus GG grows well in commercially available media for lactic acid bacteria MRS at 37° C. (98° F.) under aerobic conditions and forms white, smooth and convex colonies. Lactobacillus rhamnosus GG has the carbohydrate fermentation pattern API 50 CH genotypic. It may be that any probiotic strain of bacteria, particularly any probiotic strain of Lactobacillus rhamnosus having a carbohydrate fermentation pattern that is API 50 CH genotypic may be suitable in the composition of the invention. Lactobacillus rhamnosus GG has a survival rate of 100% at pH 4 and pH 3 after 2 hours (L. rhamnosus GG, strain sheet, Lallemand).

In embodiments of the invention, the at least one probiotic strain of bacteria comprises a probiotic strain of Pediococcus acidilactici. Pediococcus acidilactici is a facultative anaerobe bacterium. Pediococcus acidilactici exerts antagonism against pathogens through the production of lactic acid and secretion of bacteriocins, bacteria-killing compounds, known as pediocins. In embodiments of the invention, the probiotic strain of Pediococcus acidilactici is HA-524. As an example, Pediococcus acidilactici HA-524 may be supplied by Lallemand Pediococcus acidilactici HA-524 is an example of a probiotic strain that produces L(+) lactic acid and D(−) lactic acid. Pediococcus acidilactici HA-524 has been deposited at the Belgian Co-ordinated Collections of Micro-organisms under deposit number LMG S-30610. Pediococcus acidilactici HA-524 has a cell morphology comprising: gram-positive, coccoids in groups by pairs or tetrads and are not chain forming, non-motile and non-spore-forming. Pediococcus acidilactici HA-524 is a facultative anacrobe that grows well on MRS media incubated at 37 and 45° C. It is also viable at higher temperatures up to 65° C. Pediococcus acidilactici HA-524 has the carbohydrate fermentation pattern API 50 CH genotypic. It may be that any probiotic strain of bacteria, particularly any probiotic strain of Pediococcus acidilactici having a carbohydrate fermentation pattern that is API 50 CH genotypic may be suitable in the composition of the invention. In acidic conditions, Pediococcus acidilactici HA-524 shows a survival rate of 84% (pH4) after 2 hours. Pediococcus acidilactici HA-524 is able to grow in a bile solution concentrated up to 0.3% (P. acidilactici HA-524, strain sheet, Lallemand).

In embodiments of the invention, the at least one probiotic strain of bacteria comprises a probiotic strain of Bifidobacterium longum. Bifidobacterium longum is an important organism involved in the maintenance of the human gastrointestinal tract. Its presence is associated with health benefits by normalizing gastro-intestinal transit, inhibiting the growth of harmful bacteria, and also by boosting the immune system. In embodiments of the invention, the probiotic strain of Bifidobacterium longum is Rosell-175, alternatively referred to as R0175. As an example, Bifidobacterium longum Rosell-175 may be supplied by Lallemand. Bifidobacterium longum Bifidobacterium longum Rosell-175 is an example of a bacterial strain the produces lactic acid and acetate. Rosell-175 has been deposited at the Collection Nationale de Cultures de Microorganismes under deposit number I-3470, CNCM. Bifidobacterium longum Rosell-175 has a cell morphology comprising: rods, non-sporulating, non-motile, isolated or in pairs, gram-positive, heterofermentative, strict anacrobe. Bifidobacterium longum Rosell-175 grows well in commercially available media for lactic acid bacteria (RCM, Reinforced Clostridial Medium) at 37° C. (98° F.) under anaerobic conditions and forms small white colonies. Bifidobacterium longum Rosell-175 has the carbohydrate fermentation pattern API 50 CH genotypic. It may be that any probiotic strain of bacteria, particularly any probiotic strain of Bifidobacterium longum having a carbohydrate fermentation pattern that is API 50 CH genotypic may be suitable in the composition of the invention. In acidic conditions, Bifidobacterium longum Rosell-175 shows a survival rate of 80% (pH4) and 64% (pH3) after 2 hours and a good survival to bile.

In embodiments of the invention, the at least one probiotic strain of bacteria comprises a probiotic strain of Lactobacillus reuteri. Lactobacillus reuteri is a gram-positive lactic acid bacterium. It is a natural component of the intestinal, oral and vaginal human microflora. In embodiments of the invention, the probiotic strain of Lactobacillus reuteri is HA-188. As an example, Lactobacillus reuteri HA-188 may be supplied by Lallemand. Lactobacillus reuteri HA-188 is an example of a probiotic strain that produces L(+) lactic acid and D(−) lactic acid. Lactobacillus reuteri HA-188 has been deposited at the Belgian Co-ordinated Collections of Micro-organisms under deposit number LMG S-24821. Lactobacillus reuteri HA-188 has a cell morphology comprising: rods, non-sporulating, non-motile, gram-positive, obligately heterofermentative, microaerophilic; isolated or tends to form pairs. Lactobacillus reuteri HA-188 grows well in commercially available media for lactic acid bacteria at 37° C. (98° F.) and forms white-cream, flat, mat, irregular, round colonies. Lactobacillus reuteri HA-188 has the carbohydrate fermentation pattern API 50 CH genotypic. It may be that any probiotic strain of bacteria, particularly any probiotic strain of Lactobacillus reuteri having a carbohydrate fermentation pattern that is API 50 CH genotypic may be suitable in the composition of the invention. In acidic conditions (pH 4), Lactobacillus reuteri HA-188 doesn't show bacterial loss after 3 hours at 37° C. Lactobacillus reuteri HA-188 is able to grow in a bile solution concentrated up to 1% (L. reuteri HA-188, strain sheet, Lallemand). L. reuteri HA-188 is able to produce hydrogen peroxide, which inhibits growth of pathogens and undesirable microorganisms. It may be that any probiotic strain of bacteria, particularly any probiotic strain of Lactobacillus reuteri that produces hydrogen peroxide, may be suitable in the composition of the invention.

In embodiments of the invention, the compositions comprise at least one a probiotic strain of Lactobacillus rhamnosus or Lactobacillus reuteri, or combination thereof.

In embodiments of the invention, the compositions comprise at least one probiotic strain of bacteria comprising a probiotic strain of Pediococcus acidilactici or Bifidobacterium longum or mixtures thereof. It may be that the compositions comprise a probiotic strain of Pediococcus acidilactici or Bifidobacterium longum or mixtures thereof in addition to at least one a probiotic strain of Lactobacillus rhamnosus or Lactobacillus reuteri, or combination thereof. It may be that the compositions comprise a probiotic strain of Pediococcus acidilactici or Bifidobacterium longum or mixtures thereof instead of Lactobacillus rhamnosus or Lactobacillus reuteri, or combination thereof.

In embodiments of the invention, the at least one probiotic strain of bacteria comprises a probiotic strain of each of Lactobacillus rhamnosus, Lactobacillus reuteri, Pediococcus acidilactici and Bifidobacterium longum.

Alternatively, or additionally, any one of the probiotic strains listed above may be supplied as postbiotics, for example in tyndallized form, and the composition may comprise one or more of these strains in tyndallized form.

In preferred embodiments, the microbes specified as part of the various aspects of the invention are provided in a viable or substantially viable form. Typically this may be in a freeze dried or otherwise preserved form in which metabolic activity is low but wherein a material proportion of the cells retain the ability to increase their metabolic activity and optionally multiply under appropriate conditions.

### Postbiotic Strains:

The compositions of the present invention, particularly the compositions according to the first aspect of the invention, comprise at least one postbiotic strain of bacteria. The postbiotic strain may prepared by killing or deactivating a live bacterial strain. For example, the bacteria strain is killed by a heat treatment, also referred to as tyndallization. Preferably, the postbiotic strain of bacteria is heat treated (i.e. tyndallized) such that the bacterium is inactivated and the cell wall remains intact.

Preferably, the at least one postbiotic strain comprises a postbiotic strain of Lactobacillus acidophilus or Lactobacillus plantarum, or combination thereof. Preferably, the postbiotic strain of Lactobacillus acidophilus or Lactobacillus plantarum, or combination thereof are tyndallized.

Preferably, the at least one postbiotic strain of Lactobacillus acidophilus is HA-122.

Preferably, the at least one postbiotic strain of Lactobacillus plantarum is HA-119.

Preferably the at least one postbiotic strain is a lactic acid producing strain of bacteria. Suitable bacteria include but are not limited to Lactobacillus acidophilus. and Lactobacillus plantarum.

Preferably, the composition comprises at least one strain of an immunobiotic lactic acid bacterium. Lactobacillus plantarum is a suitable example of an immunobiotic lactic acid bacterium.

Preferably, the composition of the present invention comprises a combination of at least one probiotic strain of bacteria and at least one postbiotic strain of bacteria. Preferably, the at least one probiotic strain of bacteria is selected from the strains of bacteria list in the above section entitled “Probiotic strains”. The composition of the present invention may comprise one postbiotic strain of Lactobacillus acidophilus and at least one probiotic strain of Lactobacillus rhamnosus. The composition of the present invention may comprise at least one postbiotic strain of Lactobacillus acidophilus and at least one probiotic strain of Lactobacillus reuteri. Preferably, the composition of the present invention comprises at least one postbiotic strain of Lactobacillus acidophilus and at least one probiotic strain of Lactobacillus rhamnosus and at least one probiotic strain of Lactobacillus reuteri. Most preferably, the at least one postbiotic strain of Lactobacillus acidophilus is HA-122, the at least one probiotic strain of Lactobacillus rhamnosus is GG and the at least one probiotic strain of Lactobacillus reuteri is HA-188. Optionally, the composition may further comprise a probiotic strain of Pediococcus acidilactici or Bifidobacterium longum or mixtures thereof. Optionally, the composition may further comprise a postbiotic strain of Lactobacillus plantarum. Most preferably, the at least one probiotic strain of Pediococcus acidilactici is HA-524. Most preferably, the at least one probiotic strain of Bifidobacterium longum is Rosell-175. Most preferably, the at least one postbiotic strain of Lactobacillus plantarum is HA-119.

### Microbial Strains

Compositions according to the invention may in certain embodiments comprise one or more preferred strains as shown in the table below.

| | Microbial species | Preferred strain |
|---|---|---|
| Probiotic | Lactobacillus rhamnosus | GG |
| | Pediococcus acidilactici | HA-524 |
| | Bifidobacterium longum | Rosell-175 |
| | | (R0175) |
| | Lactobacillus reuteri | HA-188 |
| Postbiotic | Lactobacillus acidophilus | Tyndallized HA-122 |
| | Lactobacillus plantarum | Tyndallized HA-119 |

According to certain embodiments, one or more of the microbial species may in an optional embodiment comprise an equivalent, corresponding, similar or derivative of the strain listed above. For example, it may comprise a redeposit of one of the strains listed above. It may be a strain which is isolated from the same or similar source as one of the strains listed above, or it may be a strain for which one of the strains listed above is an ancestral strain, that is to say it may be a strain which was been obtained by natural or artificial means from one of the specified strains.

In embodiments of the invention, the probiotic strains listed above may alternatively be supplied as postbiotics, for example in tyndallized form, and the composition may comprise one or more of these strains in tyndallized form. In embodiments of the invention, the postbiotic strains listed above may alternatively be supplied in live, viable form, that is as a probiotic, and the composition may comprise one or more of these strains in live form.

The taxonomy change for Lactobacillus was officially published in the International Journal of Systematic and Evolutionary Microbiology in April 2020 (Zheng, J. et al. (2020). A taxonomic note on the genus Lactobacillus: Description of 23 novel genera, emended description of the genus Lactobacillus Beijerinck 1901, and union of Lactobacilluseae and Leuconostocaceae. International Journal of Systematic and Evolutionary Microbiology, 70: 2782-2858.) Consequently, new genera have been created in addition to the current Lactobacillus. It is to be noted that the species and the strains remain the same, the change only pertains to the genus.

| Previous taxonomy | Updated taxonomy |
|---|---|
| Lactobacillus acidophilus | Lactobacillus acidophilus |
| Lactobacillus delbrueckii | Lactobacillus delbrueckii |
| subsp bulgaricus | subsp bulgaricus |
| Lactobacillus gasseri | Lactobacillus gasseri |
| Lactobacillus helveticus | Lactobacillus helveticus |
| Lactobacillus brevis | Levilactobacillus brevis |
| Lactobacillus casei | Lacticaseibacillus casei |
| Lactobacillus fermentum | Limosilactobacillus fermentum |
| Lactobacillus paracasei | Lacticaseibacillus paracasei |
| Lactobacillus plantarum | Lactiplantibacillus plantarum |
| Lactobacillus reuteri | LimosiLactobacillus reuteri |
| Lactobacillus rhamnosus | Lacticaseibacillus rhamnosus |
| Lactobacillus salivarius | Ligilactobacillus salivarius |

The previous taxonomy and updated taxonomy may be used interchangeably, for example herein.

In embodiments of the invention, the composition comprises at least one probiotic strain that has been approved for human consumption. For example, the strain may be on the EFSA QPS (qualified presumption of safety list). Additionally, or alternatively, the strain may be approved for human consumption by the European Food Safety Agency or equivalent national organisation. It may be that the strain, or strains enhance immunity and/or reduce inflammation. It may be that the strain, or strains reduce inflammation of the skin. It may be that the strain, or strains improve the appearance of the skin, particularly the skin of the face. It may be that the strain, or strains improve the appearance of the hair, particularly the hair of the head.

In embodiments of the invention, the composition comprises at least one probiotic strain that does not possess any transferrable antibiotic resistance genes. It is preferred that all probiotic bacterial strains to be used in accordance with the invention have no transferrable antibiotic resistance genes.

In embodiments of the invention, the composition comprises at least one postbiotic strain that does not possess any transferrable antibiotic resistance genes. It is preferred that all postbiotic bacterial strains to be used in accordance with the invention have no transferrable antibiotic resistance genes.

In embodiments of the invention, the composition comprises at least one probiotic strain that has antimicrobial activity against at least one enteric pathogen, for example as Escherichia coli, E. coli ETEC, Pseudomonas aeruginosa, Salmonella typhimurium, Staphylococcus aureus, Staphylococcus aureus SARM, Cronobacter sakazakii, Clostridium difficile, Enterococcus faecium VRE, Klebsiella pneumonia and Helicobacter pylori.

In embodiments of the invention, the composition comprises at least one probiotic that is able to survive in acidic conditions.

In embodiments of the invention, the composition comprises at least one probiotic strain that produces lactic acid, preferably L(+) lactic acid. For example, Bifidobacterium longum as used in the invention may comprise a strain the produces lactic acid. Alternatively or additionally, the at least one probiotic strain may produce D(−) lactic acid. For example, Lactobacillus rhamnosus as used in the invention may comprise a strain which is able to produce L(+) lactic acid and optionally acetic acid. As another example, Lactobacillus reuteri or Pediococcus acidilactici as used in the invention may comprise a strain that produces L(+) lactic acid and D(−) lactic acid. In embodiments of the invention, the composition comprises at least one probiotic strain that has a carbohydrate fermentation pattern allowing growth on API 50 CH medium. The Lactobacillus rhamnosus strain GG meets these criteria. The Lactobacillus reuteri strain HA-188, the Pediococcus acidilactici strain HA-524, and the Bifidobacterium longum strain Rosell-175 also meet these criteria.

In embodiments of the invention, the at least one probiotic strain shows a high adhesion capacity to human cells. Without wishing to be bound by theory, this may provide a competitive advantage as it reduces the amounts of attachment sites available for pathogens. It may also be important for bacterial maintenance and colonization of the human gastrointestinal tract (Johnson, Brant R., and Todd R. Klaenhammer, “Impact of genomics on the field of probiotic research: historical perspectives to modern paradigms.” Antonie Van Leeuwenhoek 106.1 (2014): 141-156). Lactobacillus rhamnosus strain GG and Lactobacillus reuteri HA-188 meets this criteria.

In preferred embodiments, one or more strains of Lactobacillus rhamnosus are selected from the following table.

| Species | Strain |
|---|---|
| Lactobacillus rhamnosus | GG |
| Lactobacillus rhamnosus | R0011 |
| Lactobacillus rhamnosus | R1039 |
| Lactobacillus rhamnosus | CLR2 |
| Lactobacillus rhamnosus | CUL-63 |
| Lactobacillus rhamnosus | GPS 4111 |
| Lactobacillus rhamnosus | GPS 4211 |
| Lactobacillus rhamnosus | GPS 4214 |
| Lactobacillus rhamnosus | GPS 4234 |
| Lactobacillus rhamnosus | GPS 4432 |
| Lactobacillus rhamnosus | GR 1 |
| Lactobacillus rhamnosus | HA-111 |
| Lactobacillus rhamnosus | HA-114 |
| Lactobacillus rhamnosus | HA-500 |
| Lactobacillus rhamnosus | HN001 |
| Lactobacillus rhamnosus | IMC501 |
| Lactobacillus rhamnosus | LR6 |
| Lactobacillus rhamnosus | Lc705 |
| Lactobacillus rhamnosus | Lcr35 |
| Lactobacillus rhamnosus | Lr-04 |
| Lactobacillus rhamnosus | Lr-32 |
| Lactobacillus rhamnosus | MED26 |
| Lactobacillus rhamnosus | PXN 54 |
| Lactobacillus rhamnosus | R0011 |
| Lactobacillus rhamnosus | Rosell-11 |
| Lactobacillus rhamnosus | SD-5217 |

In preferred embodiments, one or more strains of Pediococcus acidilactici are selected from the following table.

| Species | Strain |
|---|---|
| Pediococcus acidilactici | HA-524 |
| Pediococcus acidilactici | R1001 |

In preferred embodiments, one or more strains of Bifidobacterium longum are selected from the following table.

| Species | Strain |
|---|---|
| Bifidobacterium longum | Rosell-175 |
| Bifidobacterium longum | Med 15 |
| Bifidobacterium longum | R0175 |
| Bifidobacterium longum | 35624 |
| Bifidobacterium longum | BB536 |
| Bifidobacterium longum | B1-05 |
| Bifidobacterium longum | CECT7347 |
| Bifidobacterium longum | DSM 24736 |
| Bifidobacterium longum | GPS 1405 |
| Bifidobacterium longum | HA-135 |
| Bifidobacterium longum | MM-2 |
| Bifidobacterium longum | PXN 30 |

In preferred embodiments, one or more strains of Lactobacillus reuteri are selected from the following table.

| Species | Strain |
|---|---|
| Lactobacillus reuteri | HA-188 |
| Lactobacillus reuteri | NCIMB 30242 |
| Lactobacillus reuteri | DSM 17938 |
| Lactobacillus reuteri | RC-14 |

### Level of Specified Microbial Organisms

The number of viable cells of each of the specified microbial species is conveniently expressed in terms of colony-forming units (CFUs). This may be assessed by any appropriate method. For example as outlined in Goldman, Emanuel; Green, Lorrence H Practical Handbook of Microbiology, Second Edition (Second ed.). USA: CRC Press, Taylor and Francis Group. p. 864. ISBN 978-0-8493-9365-5 (herein incorporated by reference).

According to some embodiments of the invention the total amount of bacteria present in a composition according to the invention is between about 15×109 and about 20×109 CFU at the point of manufacture, for example about 17×109 CFU at the point of manufacture. For probiotic strains CFU values may be understood as to represent the number of viable cells prior to the cells being killed, for example by tyndallisation.

In embodiments of the invention, the total amount of the at least one probiotic strain of bacteria selected from probiotic strains of Lactobacillus rhamnosus, Pediococcus acidilactici, Bifidobacterium longum, and Lactobacillus reuteri or combination thereof and the at least one postbiotic strain of bacteria may be greater than about 1×108 but less than about 20×109 CFU, at the point of manufacture, for example about 17×109 CFU at the point of manufacture.

In some embodiments, the total amount of strains of each of Bifidobacterium longum, Lactobacillus rhamnosus, Pediococcus acidilactici, Lactobacillus reuteri Lactobacillus acidophilus and Lactobacillus plantarum in a composition of the invention may be greater than about 1×108 but less than about 20×109 CFU at the point of manufacture, provided that the combined total number of CFU of all bacterial species may not exceed 20×109 CFU. Preferably, the total amount of Bifidobacterium longum may be 5.40×109 CFU at the point of manufacture. Preferably, the total amount of Lactobacillus rhamnosus may be 5.40×109 CFU at the point of manufacture. Preferably, the total amount of Pediococcus acidilactici may be 5.40×109 CFU at the point of manufacture. Preferably, the total amount of Lactobacillus reuteri may be 0.165×109 CFU at the point of manufacture. Preferably, the total amount of Lactobacillus acidophilus may be 5×108 CFU at the point of manufacture. Preferably, the total amount of Lactobacillus plantarum may be 5×108 CFU at the point of manufacture.

It is likely that in most compositions there will also be a certain level of non-viable cells and/or cell debris. However according to certain embodiments, viable microbial cells in total make up at least 5, 10, 20, 30, 40, 50, 60, or 70% of the total weight of the composition.

After the point of manufacture, the amount of viable bacteria present in the composition will decrease owing to die-off. A convenient way of expressing this is by determining the minimum amount of bacteria present at the best before date or the minimum amount of bacteria present at the date the composition is orally administered. According to certain embodiments the total amount of the at least one probiotic strain of bacteria selected from probiotic strains of Lactobacillus rhamnosus, Pediococcus acidilactici, Bifidobacterium longum, and Lactobacillus reuteri or combination thereof and the at least one postbiotic strain of bacteria is between about 1×109 and about 15×109 CFU at the point of oral administration and/or at the best before date of the composition, for example 5.00×109 CFU.

An exemplary composition according to the present invention is shown in the table below.

| | | % of total | | |
|---|---|---|---|---|
| | | bacterial | % | |
| | | concentration | content | Content |
| | | at the point of | in | in mg/ |
| Ingredients | Strain | manufacture | g/100 g | capsule |
| Bifidobacterium longum | Rosell-175 | 33.00% | — | — |
| | (R0175) | | | |
| Lactobacillus rhamnosus | GG | 33.00% | — | — |
| Pediococcus acidilactici | HA-524 | 33.00% | — | — |
| Lactobacillus reuteri | HA-188 | 1.00% | | |
| Total bacteria content | | | 20.60% | 86.7 |
| Lactobacillus | Tyndallised | Total = | 0.80% | 3.35 |
| acidophilus | HA-122 | 1 × 10⁹ | | |
| (standardized at 150B | | tyndallized | | |
| CFU/g) | | bacteria/ | | |
| Lactobacillus plantarum | Tyndallised | capsule | 0.80% | 3.35 |
| (standardized at 150B | HA-119 | | | |
| CFU/g) | | | | |
| Ascorbic acid (eq. 12 mg | — | — | 2.90% | 12.2 |
| vitamin C/capsule) | | | | |
| Magnesium stearate | — | — | 1.00% | 4.2 |
| Potato starch | — | — | QSP | QSP |
| | | | (73.9%) | (310.2) |

According to this example, the net weight of the capsule was 420.00 mg. The capsule used was a HPMC VCap, size 1, 75 mg, transparent. The guaranteed concentration at manufacture (COA) of bacteria was 16.50×109 CFU/capsule of bacteria (inclusive of both the tyndallised and non-tyndallised strains of bacteria). The minimum concentration of bacteria expected at the end of shelf life (label claim) was 5.00×109 CFU/capsule. The expected shelf life of the exemplary composition was at least 24 months at 25° C. (as determined by LHS analytical methods).

### Other Ingredients:

Compositions according to the invention may contain one or more further ingredients. Preferably these additional ingredients are generally recognised as safe or are approved for cosmetic, food and/or pharmaceutical use.

Exemplary compositions for oral administration may contain diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxypropyl cellulose (HPC), hydroxypropyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavours, colouring agents and stabilizers may also be added for ease of fabrication and use. For example, one or more antioxidants such as ascorbic acid, and/or one or more anticaking agents such as vegetable magnesium stearate, potato starch and ground rice hull (for example, the commercially available product Ribus), may be included as additional ingredients in compositions according to the invention. In certain preferred embodiments the compositions of the invention further comprise potato starch and ascorbic acid

It has been found that, surprisingly, compositions of the invention can be effective when provided in a dosage form which lacks an enteric coating. It has been found that the preferred strains of compositions of the invention are naturally resistant to gastric acidity and that they are also stable during manufacture and storage. Further protection against stomach acid may be provided by providing the microorganisms of in compositions of the invention wherein they are provided in a matrix of potato starch and in the presence of ascorbic acid.

In embodiments of the invention, the composition comprises at least one additional ingredient selected from the list of mineral, vitamin, yeast, and prebiotics, probiotics, postbiotics and parabiotics.

In embodiments of the invention, the composition may comprise at least one vitamin. For example, the composition may comprise one, two, three or more vitamins. Preferably the at least one vitamin is selected from the list of vitamin A, vitamin C (ascorbic acid), vitamin D, vitamin E, vitamin K, vitamin B1, vitamin B2, vitamin B3, vitamin B5 (pantothenic acid), vitamin B7 (biotin), vitamin B6, vitamin B12 and vitamin B9 (folate). Most preferably, the at least one vitamin is vitamin C.

Preferably the at least one vitamin is present in an amount equivalent to the recommended daily allowance dose (RDA) of that vitamin according to age and sex. Alternatively, the at least one vitamin is present in an amount equivalent to a fraction of the recommended daily allowance dose, for example 75%, 50%, or 25%. The at least one vitamin may be present in the composition in an amount of between about 0.1 mg and 80 mg per vitamin. For example, between about 5 mg and 25 mg per vitamin. Preferably, wherein the composition comprises vitamin C, the amount of vitamin C in the composition is about 12 mg. The at least one vitamin may be present in an amount within a tolerance of between about 80-150% of the authorised range of dosage at manufacturing for vitamins.

Vitamin C is a particularly advantageous component because vitamin C contributes to normal collagen formation for the normal function of skin.

Preferably, the compositions of the invention comprise a source of zinc, for example zinc oxide or zinc-enriched yeast. A source of zinc may improve the appearance of the skin, for example by having an anti-inflammatory effect and/or in reducing the production of sebum by the skin. Preferably, the composition comprises between about 0.1 mg and about 25 mg of a source of zinc, such as between about 1 mg and about 5 mg of a source of zinc, most preferably wherein the composition comprises about 2 mg of a source of zinc.

Preferably the composition further comprises an anti-caking agent. For example, the composition may comprise a combination of vegetable magnesium stearate and potato starch.

Preferably, the composition is free from artificial colours. Preferably, the composition is free from flavourings. Preferably, the composition is gluten free.

### Dosage Forms

Compositions of the invention may optionally be provided in the form of single dosage forms. Suitable dosage forms include tablets, liquids, capsules, and sachets of powders, each containing a predetermined amount of the ingredients. Other suitable dosage forms include chewable tablets, meltable tablets (for example tablets that disintegrate on the tongue) gummies (for example chewable confectionery), chewing gum, edible paste and edible gel.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release.

In certain embodiments the composition is provided as a dosage form which is a capsule. Any suitable capsule shell may be used and the ingredients of the dosage form may be provided as powder or granules inside the capsule shell. In embodiments of the invention, the compositions of the invention are provided in a hard shell capsule. Preferably the capsule is suitable for vegetarians, that is that the capsule does not comprise any animal product. For example, the capsule may be a vegetable based capsule shell comprising hydroxypropyl methylcellulose. The capsule may be transparent.

If the compositions are not provided in a single dosage form, for example if they are provided as a powder for use as a food supplement or as a liquid to drink, they are preferably provided in a container with dosage instructions and may additionally be provided with a device (such as a spoon or scoop) for conveniently measuring the preferred dosage.

In preferred embodiments the dosage form lacks an enteric coating and comprises the specified bacteria and yeast species or preferred strains thereof in a matric of potato starch in the presence of ascorbic acid.

### Cosmetic and Therapeutic Methods

The present invention provides a cosmetic benefit to the user. For example, it improves the appearance of skin and/or hair. However in certain embodiments, certain aspects of the invention, may arguably be regarded as relating to therapeutic methods for preventing or treating a disease or disorder. It is acknowledged that in some jurisdictions, claims to methods of treating or preventing a disorder may be regarded as patentable subject matter whereas in other jurisdictions such methods may be regarded as excluded subject matter. In this regard, the applicant reserves the right to amend the claims to comply with jurisdiction-specific subject matter requirements. For example, the applicant envisages limiting any method of the invention in accordance with any embodiment to cosmetic methods or to non-therapeutic methods, or to methods which are not methods of treating the human or animal body by therapy. Similarly, the applicant envisages claiming any embodiment of any aspect of the invention in “medical use” format. For example in “Swiss form” claim format, or in the statutory first or second medical use formats provided for in the European Patent Convention, the UK Patent Act 1977 and the law of other jurisdictions.

### Cosmetic Method and Uses

### Improvements to the Appearance of Skin

Cosmetic methods and uses envisaged by the present invention include methods of improving the appearance of the skin, especially of facial skin, for example by reducing wrinkle depth and/or improving the texture and tone of the skin. The cosmetic methods and uses envisaged by the present invention may additionally or alternatively include methods of improving the appearance of the skin by having anti-wrinkle effects and/or anti-melanogenesis effects. The composition may increase hyaluronic acid production in the skin, which is an extracellular matrix associated with the retention of skin moisture. As a consequence, the compositions of the present invention may maintain or increase skin hydration.

A further improvement in the appearance of the skin may include a decrease in transepidermal water loss (TEWL). An increase in transepidermal water loss impairs the functions of the enzymes within the skin and this results in visibly dry and aged skin. High TEWL values are indicative of disturbances in the skin's barrier function and are frequently correlated with low levels of hydration of the stratum corneum.

Other improvements to the appearance of the skin may include a reduction in skin roughness or an increase in skin smoothness, a decrease in wrinkle depth, length or density, an improvement in wrinkle smoothness and/or an improvement in skin elasticity. Wrinkle formation is associated with the regulation of collagen synthesis and degradation. Photoaging is characterized by the degradation of collagen and the accumulation of abnormal elastin in the superficial dermis. A further improvement may be an improvement in erythema. The cosmetic methods and uses envisaged by the present invention may additionally or alternatively include methods of improving the appearance of skin by decreasing imperfections and problem areas associated with fatigue of the skin over time (i.e. an improvement in fatigability of the skin). Without wishing to be bound by theory, a decrease in fatigability of the skin may indicate an improvement of the cutaneous state.

The change in appearance of the skin may be exhibited in any area of the body, for example the face, scalp, neck, décolletage, back, arms, legs, torso, hands and/or feet. The appearance of the skin may be improved within regions of the body area. For example, an improvement in the appearance of the skin of the face may occur in the nose, forehead, cheeks, chin or upper lip region of the face.

An improvement in the appearance of the skin may be observed in any part of the body. However, areas where the improvement in the appearance of the skin may be particularly pronounced are the skin around the eyes, the skin of the check, neck and/or forearm.

The present invention also envisages use of compositions of the present invention in any of the cosmetic uses disclosed herein.

### Improvements to the Appearance of Hair

Cosmetic methods and uses envisaged by the present invention include methods of improving the appearance of the hair, especially an improvement in the appearance of the hair of the scalp. Examples of improvements to the appearance of the hair include, but are not limited to a decrease in hair breakage, an increase in hair thickness, a decrease in hair loss or thinning, a decrease in loss of hair pigmentation and an increase in the glossy appearance of the hair.

### Medical Methods

Therapeutic methods and uses envisaged by the present invention include the use of a composition of the invention as a medicament, in particular as a medicament in the treatment of a skin disease, most particularly a skin disease associated with ageing skin.

### Subjects to be Treated

According to the present invention, the subjects to be administered a composition according to the invention, particularly a composition according to the first aspect of the invention, are human.

In some embodiments of the invention, the subjects to be administered a composition according to the invention, particularly a composition according to the first aspect of the invention, are male. In some embodiments of the invention the subjects are female.

In some embodiments of the invention the subjects are adult. Preferably, the subject is at least 30 years old. The subject may be over 60 years old. Most preferably, the subject is between 30 and 60 years old. It may be that an improvement in the appearance of the skin may be most pronounced in subjects between the age of 30 and 60 years old.

In some embodiments, subjects may be individuals with a microbiome imbalance of the skin, caused for example, by medical treatment such as antibiotics or steroid hormones, or by dietary issues, stress or other illnesses.

### Skin to be Treated

It is envisaged by the present invention that the skin to be improved is exhibited in any area of the body, for example the face, scalp, décolletage, neck, back, arms, legs, torso, hands and/or feet. The appearance of the skin may be improved within regions of the body area.

It is envisaged by the present invention that the skin to be improved includes the back, chest (especially décolletage), neck, face (for example chin, nose, forehead, cheeks, jawline or upper lip region, and the skin around the eyes). Additionally or alternatively, it is envisaged by the present invention that the skin to be treated includes the torso, arms, legs, hands or scalp.

### Dosage Regimen

According to some embodiments a composition according of the invention may be orally administered once a day or multiple times a day, for example twice a day, three times a day or more than three times a day. In some embodiments the composition is administered at the same time each day.

Preferably, the composition may be taken in the form of a capsule, for example once a day. More preferably, the composition is taken in the form of a capsule once a day together with a meal.

According to some embodiments the composition is orally administered on a daily basis as described above, and is administered continually for multiple, consecutive days, for example each day for a week, each day for a month or each day for multiple months, such as for three months. In some embodiments the composition is administered each day indefinitely. In some embodiments the composition is administered every day until skin imperfections, particularly those associated with ageing of the skin, are no longer observed. In some embodiments the composition is orally administered on alternative days, for example every other day, or one day per week.

The dosage may be delivered as one or more capsules. One capsule or more may be taken a day, for example two or more capsules per day, three or more capsules per day, five or more capsules per day or as many as ten or more capsules per day.

In some embodiments, the composition of the invention is administered to a subject in combination with a further treatment. The further treatment may be the use of one or more of a cleanser, exfoliator or moisturiser. Alternatively or additionally the further treatment may be a medical light emitting device.

In some embodiments, the composition of the invention is administered to a subject in combination with one or more of the following: prebiotics, postbiotics, vitamins, minerals, fats, phytonutrients, and botanicals.

The composition may be orally administered over several months, for example, 3 months. It may be that the composition is orally administered each day, for example once a day, over several months. Alternatively, the composition may be orally administered intermittently over several months, for example every other day. It may be that the frequency at which the composition is orally administered decreases once an improvement is observed in the appearance of the skin.

Once the appearance of the skin has been improved, the composition may continue to be administered orally to maintain the improved appearance of the skin, or to improve the appearance of the skin still further.

### EXAMPLES

### Equipment

The skin function was measured using non-invasive measurements and the following properties were assessed according to methods known in the art: skin elasticity, hydration (water content), transepidermal water loss, sebum levels, roughness, redness, and smoothness of the skin surface.

A Courage+Khazaka Dual 580 Multi Probe Adapter System (MPA) was used for skin measurements, and was fitted with the following probes. A Cutometer (model MPA 580) was used to measure skin elasticity. A Corneometer (model CM825) was used to measure skin hydration. A Mexameter probe (model MX18) was used to measure melanin (a protective skin pigment) and erythema (skin redness). A Tewameter (model TM 300) was used to measure transepidermal water loss. A Sebumeter (Model SM 815) may be used to determine sebum levels. A Visioscan (model VC 98), is a UVA light video camera with high resolution and was used to study the skin surface directly. The images taken with the Visioscan show the structure of the skin surface such as skin smoothness (SeSm) and wrinkles (Sew). The Visioscan was used to assess scalp and hair health alongside the skin measurements. The top of the scalp was assessed with the Visioscan (VC 98) camera. The images taken were used to qualitatively assess the general condition of the scalp, hair thickness and hair colour. A Canfield's VISIA skin analysis imaging system was used to study the overall skin condition and determine properties such as skin smoothness, skin roughness, scaliness and wrinkles and to measure spots, wrinkles, texture, pores, UV spots, brown spots, red areas and porphyrins. This imaging system captures high quality, standardized facial images. All devices were manufactured and maintained according to the cosmetic industry standards for non-invasive technologies for assessing the various parameters of skin.

A Cutometer uses a suction method to determine the elasticity and firmness of the skin. A negative pressure is applied to the skin which causes it to mechanically deform by drawing the skin into an aperture within the device. After a period of time, the skin is released from the device. The penetration depth of the skin within the device is measured by an optical measuring system comprising a light source and a light receptor. Light is projected onto the skin and the light intensity returned to the light receptor varies depending on the penetration depth of the skin. The penetration depth in mm/time is measured in real time and the resistance of the skin to negative pressure (firmness) and its ability to return to its original position (elasticity) are recorded.

A Corneometer may be used to measure the hydration of the skin surface (about 10-20 μm depth of the stratum corneum). This probe allows quick measurements (about 1 s) to be taken, and also allows continuous measurements to be taken over a prolonged period of time. The probe works by measuring the capacitance of the uppermost layer of the skin, the stratum corneum. With increasing hydration, the dielectric properties of the skin change. Water has a high dielectric constant compared to most other substances. The Corneometer measures the change in the dielectric constant due to skin surface hydration changing the capacitance of a precision capacitor and is sensitive to very small changes in hydration level of the skin.

A Mexameter is a spectrometer measurement technique. The probe emits at the skin three wavelengths of light (green light of a wavelength of 568 nm, red light of a wavelength of 660 nm, and infrared light of a wavelength of 870 nm) that correspond to the different absorption rates of melanin and haemoglobin. The receiver of the probe then measures diffuse and scattered light which is reflected by the skin to determine the amount of melanin and erythema of the skin.

A Tewameter may be used to assess transepidermal water loss which is a parameter for evaluating the water barrier function of the skin. If the barrier function of the skin is damaged, this will result in an increase in water loss. The Tewameter probe is a hollow cylinder containing sensors to measure relative humidity and temperature. The probe measures the density gradient of the water evaporation from the skin which is proportional to the transepidermal water loss. Transepidermal water loss is measured in g/m2/h and can be used to measure skin functionality, in particular barrier function.

A Sebumeter may be used to determine the sebum level of the skin surface. The sebumeter contains a tape which reacts with sebum to become transparent. The tape is placed in contact with the skin to allow the tape to react to sebum and then the transparency of the tape is measured by inserting the tape into an aperture in the device and measuring the transparency by a photocell.

Full face photos may be taken with a Visia machine (Canfield) with Visioscan VC 98 imager both before and after the subject was administered a composition according to the invention. The Visia machine (Canfield) uses cross-polarized and UV lighting to record and measure surface and subsurface skin conditions. Images of sections of the skin of the face are taken with the Visia machine which can then be combined into a full face photograph. UV photography provides the most complete data set available for sun damage assessment and analysis, including UV fluorescence imaging to reveal porphyrins, spots, wrinkles, texture, pores, brown spots, red areas and UV spots. The images were assessed for visual changes in appearance of the skin, for example a reduction in skin redness, dryness, dullness, or oiliness, or a generally more healthy appearance of the skin following administration of the inventive composition.

Biomass on the skin of the face may be collected using a standard swabbing method. The forehead, nose and chin of each subject may be swabbed and analysed separately. The skin of the face may be swabbed using a collection kit comprising Geneflow Swab Collection & DNA preservation kit & Norgren wetting tube. The microbiome of the skin may be analysed by 16s rRNA analysis. Alternatively, or additionally, the microbiome of the skin may be analysed by UV imaging in which porphyrins produced by Cutibacterium acnes fluoresce under UV irradiation (near 400 nm). UV facial images may be taken before and after the subject had been orally administered a composition according to the invention. A reduction in the amount of fluorescence associated with porphyrins may be indicative of a reduction in Cutibacterium acnes present on the skin.

Bacterial and yeast strains were purchased from Lallemand (Mirabel, Canada) and, incorporated into the final formula.

### Overall Skin Condition

The Visia machine (Canfield) uses cross-polarized and UV lighting to record and measure surface and subsurface skin conditions. UV photography provides the most complete data set available for sun damage assessment and analysis, including UV fluorescence imaging to reveal porphyrins, spots, wrinkles, texture, pores, brown spots, red areas and UV spots.

16s rRNA (v1/v3 Region) Sequencing

Use of broad range 16S rRNA gene PCR as a tool for identification of bacteria is possible because the 16S rRNA gene is present in all bacteria. The 16S rRNA gene consists of highly conserved nucleotide sequences, interspersed with variable regions that are genus- or species-specific. PCR primers targeting the conserved regions of rRNA amplify variable sequences of the rRNA gene. Bacteria can be identified by nucleotide sequence analysis of the PCR product followed by comparison of this sequence with known sequences stored in a database (Jenkins, C., et al. (2012). Detection and identification of bacteria in clinical samples by 16S rRNA gene sequencing: comparison of two different approaches in clinical practice. Journal of Medical Microbiology, 61, 483-488).

### High Sensitivity C Reactive Protein

The high sensitivity C Reactive Protein (hs-CRP) levels in participants blood was also measured. hs-CRP is a protein in the blood that increases when inflammation is present. High sensitivity C-Reactive Protein is a more sensitive test than the standard CRP test and can be used to detect acute inflammation and long-term inflammation in the body. Blood was collected and analysed using a finger prick test and testing kits provided by Medichecks (in partnership with UKAS accredited laboratories).

### Self-Assessment Questionnaire

At the end of the 12 week study, participants were asked to complete a self-assessment questionnaire. The questionnaire contained questions about the appearance of the skin and hair. The participants were asked to indicate whether they strongly agreed, agreed, neither agreed nor disagreed, or disagreed with the following statements:
I feel like my skin has more of a natural glow while using this product.
My skin appears less dull and more radiant after using the test product.
After using this product, my skin looked more radiant than when I use just my normal skincare routine.
The product visibly improved the appearance of my skin.
This product reduced the fine lines and wrinkles around my eye area.
I felt like my skin took on a younger and fresher appearance.
I found that I had less hair falling out when brushing my hair.
My scalp felt less dry and itchy while using this product.
My hair felt thicker and glossier whilst using this product.
My overall wellbeing improved while using this product.
I felt less bloating whilst using this product.

### Example 1

In a trial study, a food supplement comprising Formula A was provided to a first group of participants (Group 1). In a comparative study, a food supplement comprising Formula B was provided to a second group of participants (Group 2). The participants were healthy adult females aged between 30 and 60 years that had fine lines and wrinkles, aged skin, uneven skin tone and skin prone to redness. Group 1 participants were instructed to consume 1 capsule of Formula A per day for 12 weeks, and Group 2 participants were instructed to consume 1 capsule of Formula B per day for 12 weeks. The participants were instructed to consume the capsule with any meal of the day. Participants continued with their usual skincare routine and were instructed to not make any changes to the products or routine they would usually use. The participants were forbidden from undergoing facial treatments during the 12 weeks, use any topical medications or be exposed to UV tanning devices. The participants were also forbidden from taking other food supplements during the trial.

The formulations comprised the ingredients listed in Table 1. The postbiotic strains of bacteria present in Formula B were prepared by tyndallisation. No postbiotic strains of bacteria were present in Formula A.

TABLE 1

| Formula A | Formula B |
|---|---|
| Vitamin C, 12 mg | Vitamin C, 12 mg |
| Lactobacillus rhamnosus | Lactobacillus rhamnosus |
| GG (probiotic) | GG (probiotic) |
| 1.65 × 10⁹ CFU | 1.65 × 10⁹ CFU |
| Pediococcus acidilactici, | Pediococcus acidilactici, HA-524 |
| HA-524 (probiotic) | (probiotic) |
| 1.65 × 10⁹ CFU | 1.65 × 10⁹ CFU |
| Bifidobacterium longum, | Bifidobacterium longum, Rosell-175 |
| Rosell-175 (probiotic) | (probiotic) |
| 1.65 × 10⁹ CFU | 1.65 × 10⁹ CFU |
| Lactobacillus reuteri, HA-188 | Lactobacillus reuteri, HA-188 |
| (probiotic) | (probiotic) |
| 0.05 × 10⁹ CFU | 0.05 × 10⁹ CFU |
| — | Tyndallized Lactobacillus acidophilus, |
| | HA-122 (postbiotic) at 3.35 mg |
| — | Tyndallized Lactobacillus plantarum, |
| | HA-119 (postbiotic) at 3.35 mg |

The Courage+Khazaka Dual 580 MPA was used to study the participants' skin at the beginning of the trial and after 12 weeks of consuming Formula A or Formula B.

The results of the trial studies on skin elasticity, wrinkle depth and skin smoothness are provided in Table 2. Participants in Group 1 consumed Formula A while participants in Group 2 consumed Formula B. It was surprisingly found that those participants who had consumed Formula B (the formulation comprising postbiotic strains of bacteria) showed a significant improvement in elasticity of the skin in the eye, cheek and forearm regions compared to the participants who consumed Formula A. The participants who consumed Formula B also had a greater reduction in wrinkle depth in the cheek area and a greater improvement in skin smoothness of the cheek compared to those participants who consumed Formula B.

TABLE 2

| | Group 1 | Group 2 | |
|---|---|---|---|
| | (Formula A) | (Formula B) | |
| | Average | Average | % Percentage |
| | Improvement | Improvement | Change |
| Elasticity | | | |
| Eye | 0.05 | 5.98 | 11860% |
| Cheek | 0.03 | 5.85 | 19400% |
| Forearm | 0.06 | 8.93 | 14783% |
| Wrinkle Depth | | | |
| Cheek | −50.95 | −63.34 | 24% |
| Skin Smoothness | | | |
| Cheek | −68 | −74 | 9% |

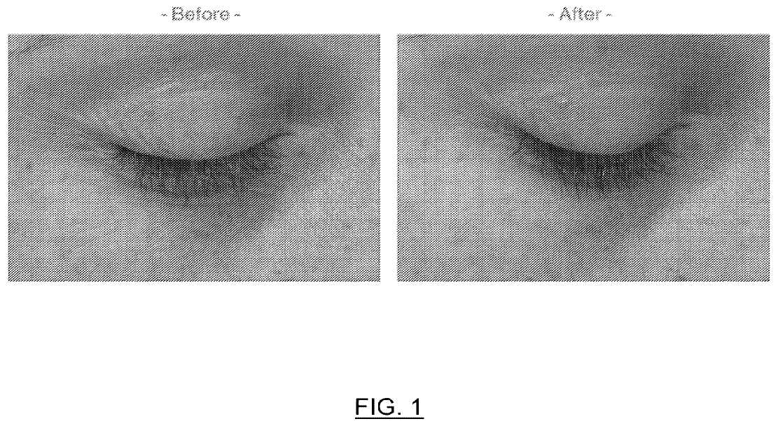

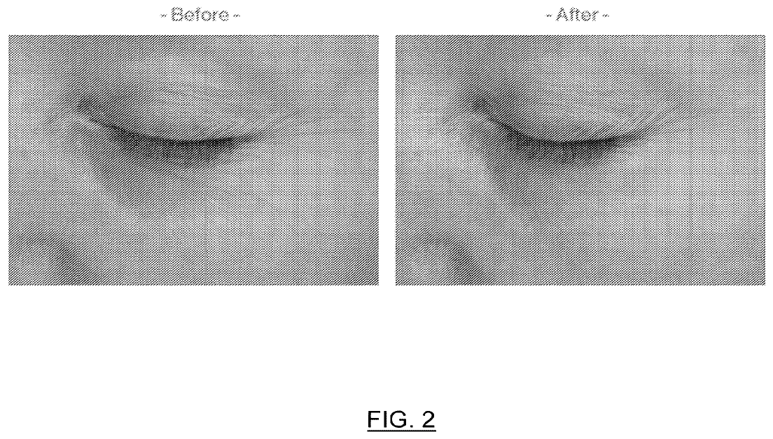

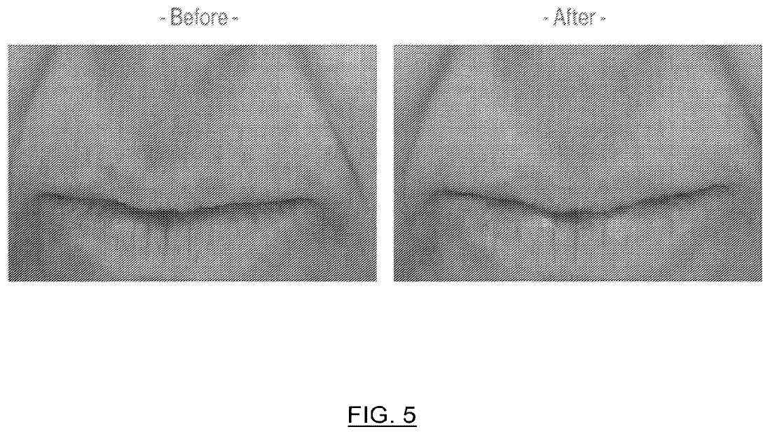

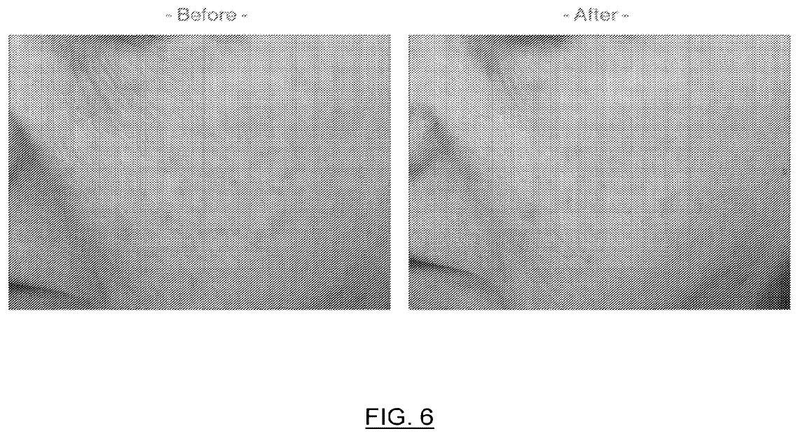

FIG. 1, FIG. 2, FIG. 5 and FIG. 6 are photographs of some participants of Group 2 taken before and after the 12 week trial. In general, a reduction in wrinkle depth and the number of wrinkles was observed at the end of the 12 week trial.

A Mexameter was used to measure the amount of erythema and melanin of the skin of participants in Group 2 before and after consuming Formula B in the 12 week trial. The results are shown in Table 3. The group mean average of erythema (skin redness) and mean average of melanin decreased after 12 weeks of consuming Formula B. 94% of participants in Group 2 experienced a reduction in erythema in at least one of area of the skin. 90% of participants in Group 2 experienced a reduction in erythema in at least one area of the face. 74% of participants in Group 2 experience a reduction in erythema in the cheek area. 68% of participants in Group 2 experienced a reduction in erythema in the forehead area. 48% of participants in Group 2 experienced a reduction in erythema around the eye area. 97% of participants in Group 2 experienced a reduction in melanin in at least one area. 90% of participants in Group 2 experienced a reduction in melanin in at least one area of the face. 77% of participants in Group 2 experienced a reduction in melanin in the forehead area. 74% of participants in Group 2 experienced a reduction in melanin around the eye area. 26% of participants in Group 2 experienced a reduction in melanin in the cheek area.

TABLE 3

| Erythema | Mean | Mean | % Percentage |
|---|---|---|---|
| (Redness) | Before | After | change |
| Eye | 313.1 | 292.06 | −7% |
| Cheek | 366.8 | 329.64 | −10% |
| Forearm | 281.5 | 256.9 | −9% |
| Melanin | | | |
| Eye | 255.8 | 244.26 | −5% |

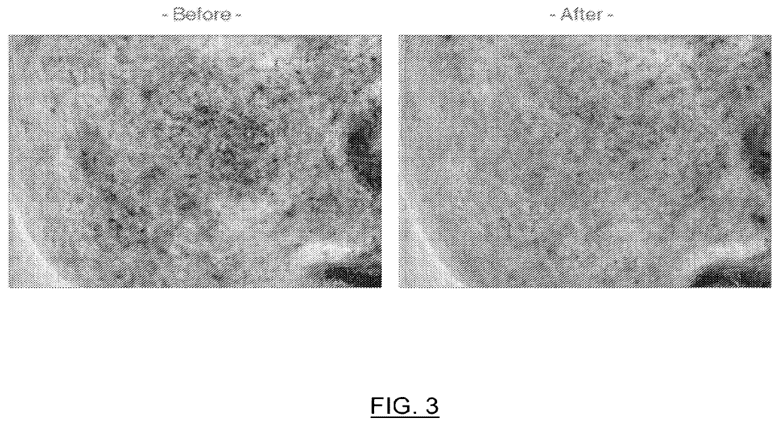

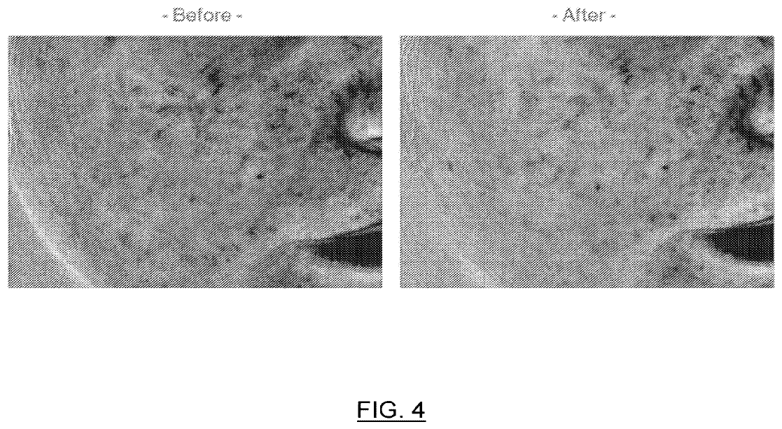

FIG. 3 and FIG. 4 are images of the cheek area taken of participants in Group 2. A reduction in skin redness (erythema) was observed at the end of the 12 week trial study.

The high sensitivity C Reactive Protein levels of 18 participants in Group 2 (having consumed Formula B) was also measured. The results are shown in Table 4. Those participants having the highest hs-CRP levels at the beginning of the trial (participant 1, 9 and 18) experienced a significant reduction in hs-CRP scores (a reduction of 71%, 20% and 90% respectively) by the end of the trial, indicating a reduction in inflammation in the body.

TABLE 4

| | | hs-CRP (mg/L) | |
|---|---|---|---|
| Participant | Age | Week 1 | Week 12 |
| 1 | 33 | 6.0 | 1.7 |
| 2 | 31 | 1.1 | 1.0 |
| 3 | 39 | 0.5 | 0.36 |
| 4 | 42 | 0.3 | 0.3 |
| 5 | 31 | 0.3 | 0.3 |
| 6 | 51 | 0.7 | 0.54 |
| 7 | 51 | 2.7 | 2.4 |
| 8 | 58 | 1.0 | 0.8 |
| 9 | 55 | 5.7 | 4.57 |
| 10 | 46 | 0.4 | 0.4 |
| 11 | 51 | 0.3 | 0.3 |
| 12 | 50 | 1.3 | 1.3 |
| 13 | 40 | 0.7 | 1.1 |
| 14 | 41 | 0.3 | 0.3 |
| 15 | 32 | 1.8 | 1.0 |
| 16 | 31 | 0.4 | 0.4 |
| 17 | 34 | 1.5 | 2.9 |
| 18 | 58 | 9.9 | 0.92 |

The results of the self-assessment questionnaire revealed that 59% of participants in Group 2 agreed that their skin had a more natural glow during the trial. 62% of participants in Group 2 agreed that their skin appeared less dull and more radiant at the end of the trial. 62% of participants in Group 2 agreed that the appearance of their skin had visibly improved by the end of the trial. 59% of participants in Group 2 agreed the condition of their skin had visibly improved at the end of the trial. 55% of participants in Group 2 agreed that their skin felt more balanced by the end of the trial. 55% of participants in Group 2 agreed that fine lines and wrinkles around their eye area reduced by the end of the trial. 52% of participants in Group 2 felt more confident in their own skin after the 12 weeks.

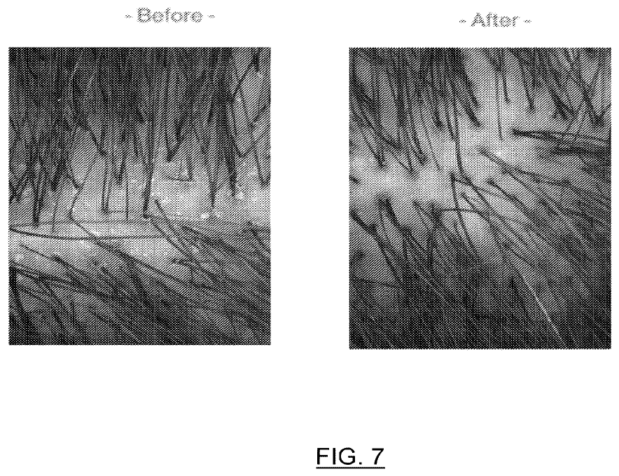

The hair and scalp health of the participants in Group 2 (who had consumed Formula B) was also assessed before and after the study trial using a Visioscan (VC 98) camera. The Visioscan images were used to make a qualitative assessment of the scalp and hair health. In general, the scalp health appeared improved, hair thickness had improved and the colour of the hair appeared less grey at the end of the study. In personal testimonials obtained for each participant in Group 2, participants commented on positive changes with their hair health during the study. FIG. 7 are photographs of the hair and scalp of a participant in Group 2 taken before and after the 12 week trial. The scalp appeared less dry and there was a reduction in flaking at the end of the 12 week trial.

The volume, length and grade of eyelashes of the participants of Group 2 was assessed using the Canfield's VISIA skin analysis machine. It was surprisingly found that the eyelash volume, length and/or grade improved by the end of the study, compared to at the start of the study. The eyelash quality measurements of 5 participants in Group 2 are provided in Table 5.

TABLE 5

| | | Eyelash | Eyelash |
|---|---|---|---|
| | | Before | After |
| HA228SYB6 | Average length | 6.2 | 6.5 |
| | Volume | 56.94 | 57.14 |
| | Grade | 2 | 2 |
| SC212SYB6 | Average length | 2.9 | 3.4 |
| | Volume | 42 | 49 |
| | Grade | 5 | 4 |
| TP218SYB6 | Average length | 3.5 | 3.8 |
| | Volume | 49 | 51 |
| | Grade | 4 | 4 |
| AV232SYB6 | Average length | 3.5 | 3.5 |
| | Volume | 41 | 44.2 |
| | Grade | 5 | 4 |
| HP211SYB6 | Average length | 3.4 | 3.4 |
| | Volume | 41 | 43.5 |
| | Grade | 5 | 4 |

* Lashes are graded from 1 to 5, a decrease in grade is considered an improvement. Length and volume improved overall in these 4 participants.

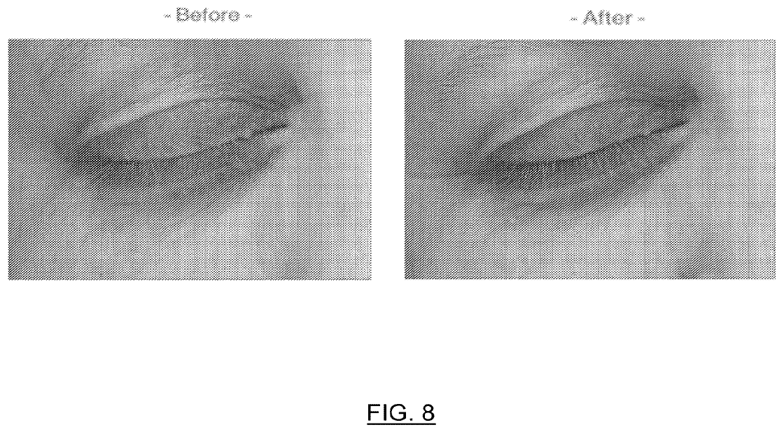

FIG. 8 are photographs of the eyelashes of participant SC212SYB6 of Table 5. An improvement in eyelash length, volume and grate was observed at the end of the 12 week study trial for that participant.

The results of one participant (aged 58 years) of Group 2 are shown in Table 6. It was found that this participant experienced improvements in all parameters measured except for wrinkle depth in the forehead area.

TABLE 6

| | Eye % | Cheek % | Forehead % |
|---|---|---|---|
| | Change | Change | Change |
| Wrinkle depth | 6.69 | 7.95 | −8.46 |
| Skin Smoothness | 2.29 | 32.81 | 4.70 |
| Elasticity | 121.34 | 24.04 | 47.29 |
| TEWL | 25.39 | 39.93 | 40.38 |
| Hydration | 17.84 | 7.10 | 21.78 |
| Erythema | 13.06 | 36.52 | 7.33 |
| Melanin | 11.28 | 5.04 | 4.75 |
| | Before | After | |
| hs-CRP | 9.9 | 0.92 | |

Whilst the present invention has been described and illustrated with reference to particular embodiments, it will be appreciated by those of ordinary skill in the art that the invention lends itself to many different variations not specifically illustrated herein. By way of example only, certain possible variations will now be described.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

What is claimed is:

1. A composition, comprising:
at least one probiotic strain of bacteria selected from the group consisting of probiotic strains of Lactobacillus rhamnosus, Pediococcus acidilactici, Bifidobacterium longum, and Lactobacillus reuteri, and combinations thereof; and
at least one postbiotic strain of bacteria.

2. The composition according to claim 1, wherein the at least one postbiotic strain of bacteria is tyndallized.

3. The composition according to claim 1, wherein the at least one postbiotic strain of bacteria comprises a postbiotic strain of Lactobacillus acidophilus or Lactobacillus plantarum, or combination thereof.

4. The composition according to claim 1, wherein the at least one probiotic strain of bacteria comprises a probiotic strain of Lactobacillus rhamnosus, Lactobacillus reuteri, Pediococcus acidilactici, Bifidobacterium longum, or combinations thereof and the at least one postbiotic strain of bacteria comprises a postbiotic strain of Lactobacillus acidophilus, Lactobacillus plantarum, or combinations thereof.

5. The composition according to claim 1, wherein the at least one postbiotic strain of bacteria comprises at least one of a postbiotic strain of Lactobacillus acidophilus wherein the postbiotic strain of Lactobacillus acidophilus is HA-122, and a postbiotic strain of Lactobacillus plantarum wherein the postbiotic strain of Lactobacillus plantarum is HA-119.

6. The composition according to claim 1, wherein the at least one probiotic strain of bacteria comprises a probiotic strain of Lactobacillus rhamnosus wherein the probiotic strain of Lactobacillus rhamnosus is GG, a probiotic strain of Pediococcus acidilactici wherein the probiotic strain of Pediococcus acidilactici is HA-524, a probiotic strain of Bifidobacterium longum wherein the probiotic strain of Lactobacillus reuteri is HA-188.

7. The composition according to claim 1, further comprising at least one of:
a vitamin selected from the group consisting of vitamin A, vitamin C, vitamin D, vitamin E, vitamin K, vitamin B1, vitamin B2, vitamin B3, vitamin B5 (pantothenic acid), vitamin B7 (biotin), vitamin B6, vitamin B12 and vitamin B9 (folate);
a yeast,
fungi,
enzyme,
botanical,
mineral,
prebiotic molecule, compound or composition; and
a postbiotic molecule, compound, composition, cell or cell component.

8. The composition according to claim 7, wherein the composition comprises vitamin C, preferably in an amount between about 0.1 mg and 80 mg such as between about 5 mg and 25 mg of vitamin C, most preferably wherein the composition comprises about 12 mg of vitamin C.

9. The composition according to claim 4, wherein the total amount of strains of Bifidobacterium longum in the composition is greater than about 1×108 but less than about 20×109 CFU at the point of manufacture, the total amount of strains of Lactobacillus rhamnosus in the composition is greater than about 1×108 but less than about 20×109 CFU at a point of manufacture, the total amount of strains of Pediococcus acidilactici in the composition is greater than about 1×108 but less than about 20×109 CFU at the point of manufacture, the total amount of strains of Lactobacillus reuteri in the composition is greater than about 1×108 but less than about 20×109 CFU at the point of manufacture, the total amount of strains of Lactobacillus acidophilus in the composition is greater than about 1 mg but less than about 10 mg at the point of manufacture, and the total amount of strains of Lactobacillus plantarum in the composition is greater than about 1 mg but less than about 10 mg at the point of manufacture.

10. The composition according to claim 1, in the form of a capsule, powder or liquid for oral administration.

11. A method for improving the appearance of the skin and/or hair of a subject, wherein the subject is a human, the method comprising:
(i) orally administering to the subject the composition of claim 1, and,
(ii) repeating step (i) until a cosmetically beneficial improvement in the appearance of the skin and/or hair of the subject has occurred.

12. The method according to claim 11, wherein the composition is orally administered in step (i) in the form of a tablet, capsule, powder, liquid, food, or drink.

13. The method according to claim 11, wherein the composition is orally administered with a meal.

14. The method according to claim 11, wherein the subject is female or male.

15. The method according to claim 11, wherein the subject is between 30 and 60 years old.

16. The method according to claim 11, wherein the method further comprises:
(iii) further repeating step (i) in order to maintain the cosmetically beneficial improvement in the appearance of the skin and/or hair of the subject, for example step (i) is repeated once a day for several months, for example three months.

17. The method according to claim 11, wherein the cosmetically beneficial improvement of the skin is one or more improvement selected from the group consisting of: increased hydration, a reduction in transepidermal water loss, a reduction in skin roughness or an increase in skin smoothness, a reduction in scaling of the skin, a reduction in the appearance of wrinkles and fine lines, a reduction in wrinkle depth, length or density, an increase in elasticity, an improvement in erythema, a reduction in melanogenesis such that the skin has a more even colour tone, an increase in skin firmness, a decrease in fatigability, a normalisation of sebum secretion, and a decrease in spots, spot breakouts, or skin imperfections.

18. The method according to claim 11, wherein the cosmetically beneficial improvement of the hair is one or more improvement selected from the group consisting of: a decrease in hair breakage, an increase in hair thickness, a decrease in hair loss or thinning, a decrease in the loss of hair pigmentation, and an increase in glossy appearance of the hair.

19. A method of altering the microbiome of the skin of a subject, wherein the subject is a human, the method comprising:
(i) orally administering to the subject the composition according to claim 1, and,
(ii) repeating step (i) until the microbiome of the skin of the subject has altered.

20. The method according to claim 16, wherein the skin is the skin of the face, scalp, neck, décolletage, back, arms, legs, torso, hands and/or feet of the subject, preferably wherein the skin is the skin of the face of the subject.
